(19) United States
(12) Patent Application Publication
(10) Pub. No.: US 2022/0199252 A1
(43) Pub. Date: Jun. 23, 2022

(54) INTELLIGENT SECURE NETWORKED HEALTH MESSAGING SYSTEMS AND METHODS

(71) Applicant: Electronic Caregiver, Inc., Las Cruces, NM (US)

(72) Inventors: Anthony Dohrmann, El Paso, TX (US); Bryan John Chasko, Las Cruces, NM (US); Samuel Blake, Las Cruces, NM (US)

(21) Appl. No.: 17/693,151

(22) Filed: Mar. 11, 2022

Related U.S. Application Data
(63) Continuation-in-part of application No. 16/169,760, filed on Oct. 24, 2018.
(60) Provisional application No. 62/618,550, filed on Jan. 17, 2018.

(51) Int. Cl. G16H 40/67; G06F 9/451; G16H 10/60

(52) U.S. Cl. CPC G16H 40/67; G16H 10/60; G06F 9/453

(57) ABSTRACT

Exemplary embodiments provided herein include an intelligent secure networked health messaging system including a data retention system and a health analytics system, the health analytics system performing asynchronous processing with a user's computing device, a web services layer providing access to the data retention and the health analytics system, a batching service, wherein an application server layer transmits a request to the web services layer for data, the request processed by the batching service transparently to the user, the request processed by the batching service transparently to the user such that the user can continue to use a user-facing unity application without disruption, the application server layer including a high speed data corridor established between the application server layer and the user's computing device that provides the user-facing unity application that accesses the data retention and the health analytics system through the web services layer, and performs processing based on user interaction with the user-facing unity application.

### CROSS-REFERENCE TO RELATED APPLICATIONS

The present continuation-in-part application claims the priority benefit of U.S. Non-Provisional patent application Ser. No. 16/169,760 filed on Oct. 24, 2018 titled, “Computing Devices with Improved Interactive Animated Conversational Interface Systems,” which in turn claims the priority benefit of U.S. Provisional Patent Application Ser. No. 62/618,550 filed on Jan. 17, 2018 titled, “Interactive Animated Conversational Interface System,” the disclosures of both of these applications incorporated by reference in their entireties.

### FIELD OF THE TECHNOLOGY

The present technology relates generally to secure health messaging, and more particularly, but not by limitation, to systems and methods for secure health messaging that allow modular subsystem isolation, as well as latency remediation and improved user experiences.

### SUMMARY

Exemplary embodiments provided herein include an intelligent secure networked health messaging system configured by at least one processor to execute instructions stored in memory, the system including a data retention system and a health analytics system, the health analytics system performing asynchronous processing with a user's computing device, a web services layer providing access to the data retention and the health analytics system, a batching service, wherein an application server layer transmits a request to the web services layer for data, the request processed by the batching service transparently to the user, the request processed by the batching service transparently to the user such that the user can continue to use a user-facing unity application without disruption, the application server layer including a high speed data corridor established between the application server layer and the user's computing device that provides the user-facing unity application that accesses the data retention and the health analytics system through the web services layer, and performs processing based on user interaction with the user-facing unity application, the user-facing unity application configured to execute instructions including, transmitting an interactive conversational user interface to the user's computing device.

Further exemplary embodiments include the interactive conversational user interface configured to activate a patient tutorial, display a medication schedule, display a vital sign, and display an external weather environment similar to a patient's actual external weather environment. The interactive conversational user interface may also be configured with a visual sensor in some cases for facial recognition of a patient and/or for facial recognition of a patient's facial changes.

In most exemplary embodiments, the interactive conversational user interface may be configured not to receive verbal input and/or not to receive verbal input unless a patient requests help. The interactive conversational user interface may be configured to contact an emergency service.

The interactive conversational user interface, in various exemplary embodiments, may be configured to link an account to the system and/or to display a special pin code, sometimes for entry of information regarding a health matter or a personal matter. The interactive conversational user interface may also be configured to receive information from a blood pressure monitor, a glucometer, a pulse oximeter, a pro health hub and/or a thermometer over a network.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a schematic diagram of an exemplary computing architecture that includes a system constructed in accordance with the present disclosure.

FIGS. 2-74 show exemplary interactive conversational user interfaces.

### DETAILED DESCRIPTION

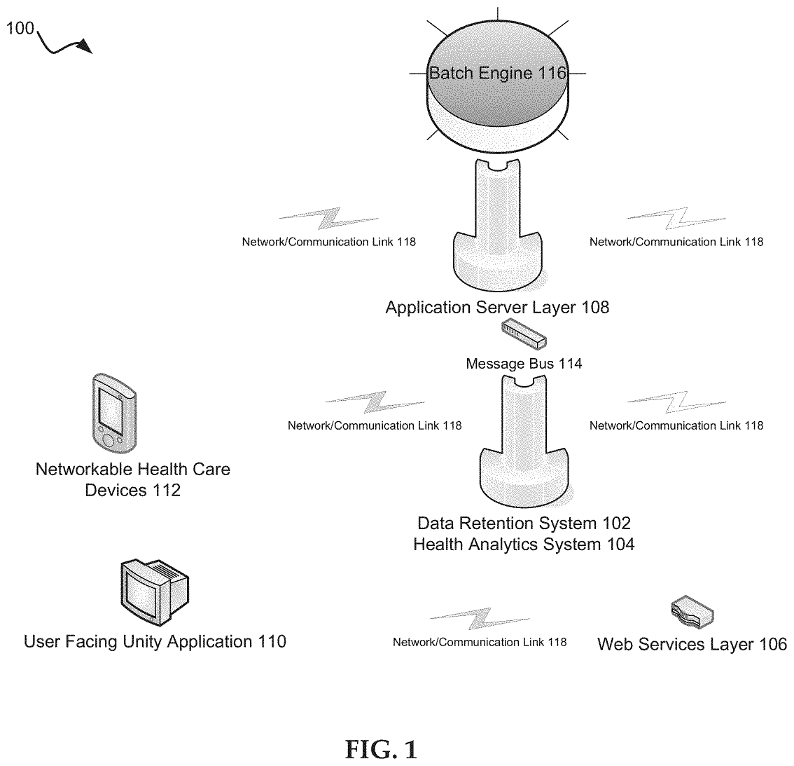

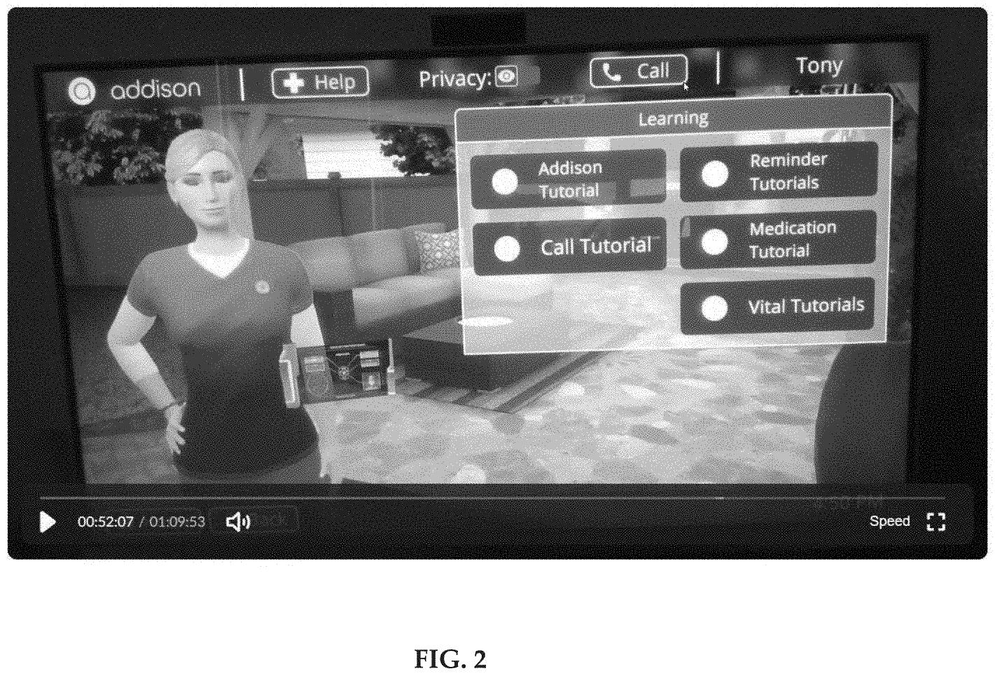

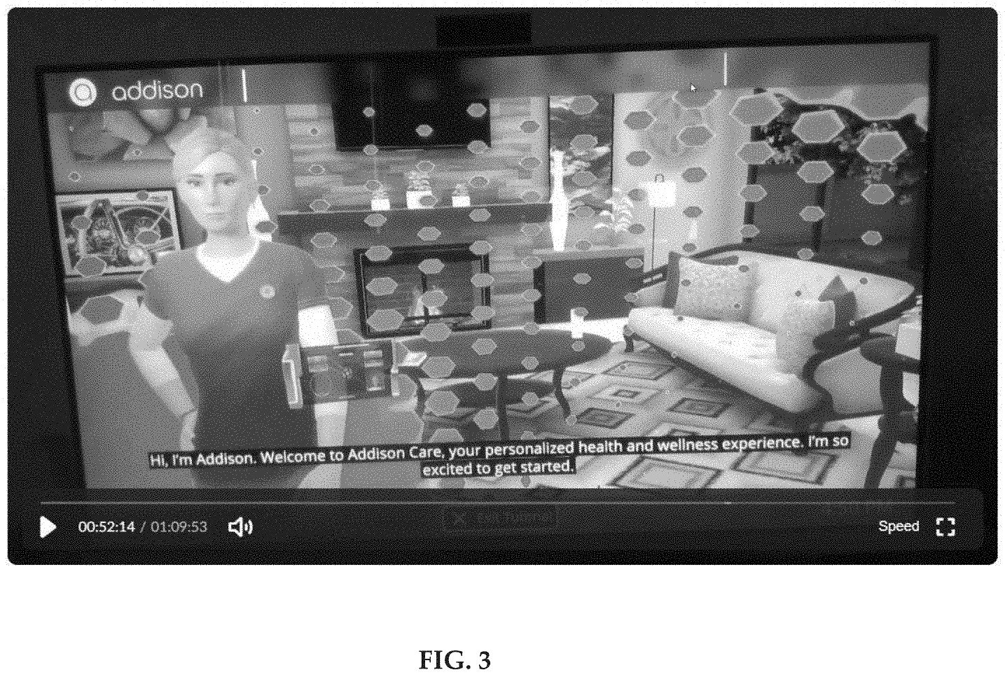

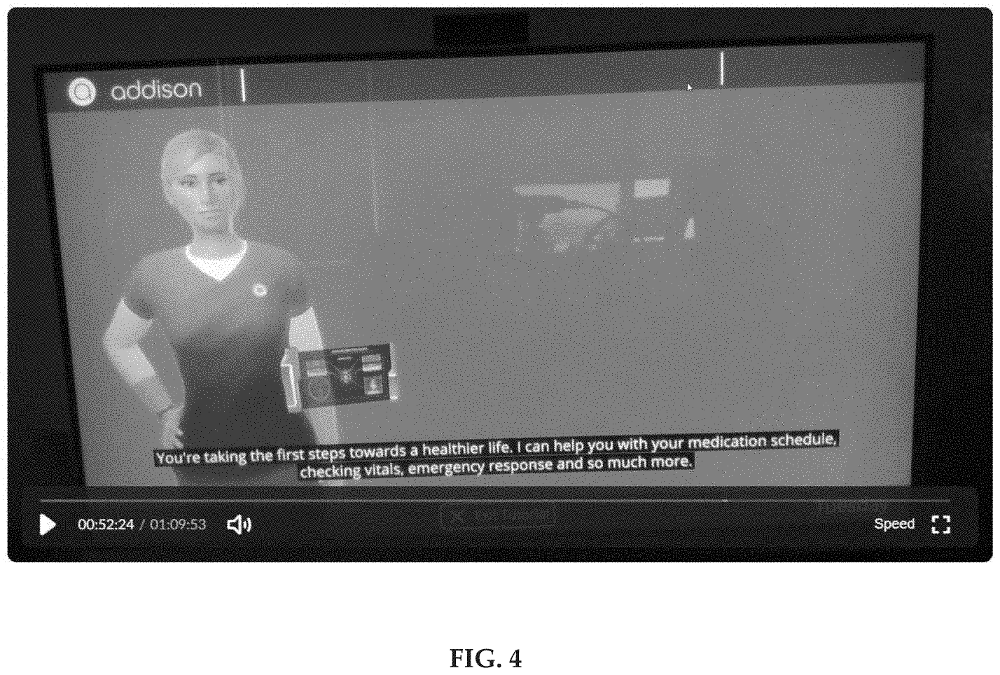

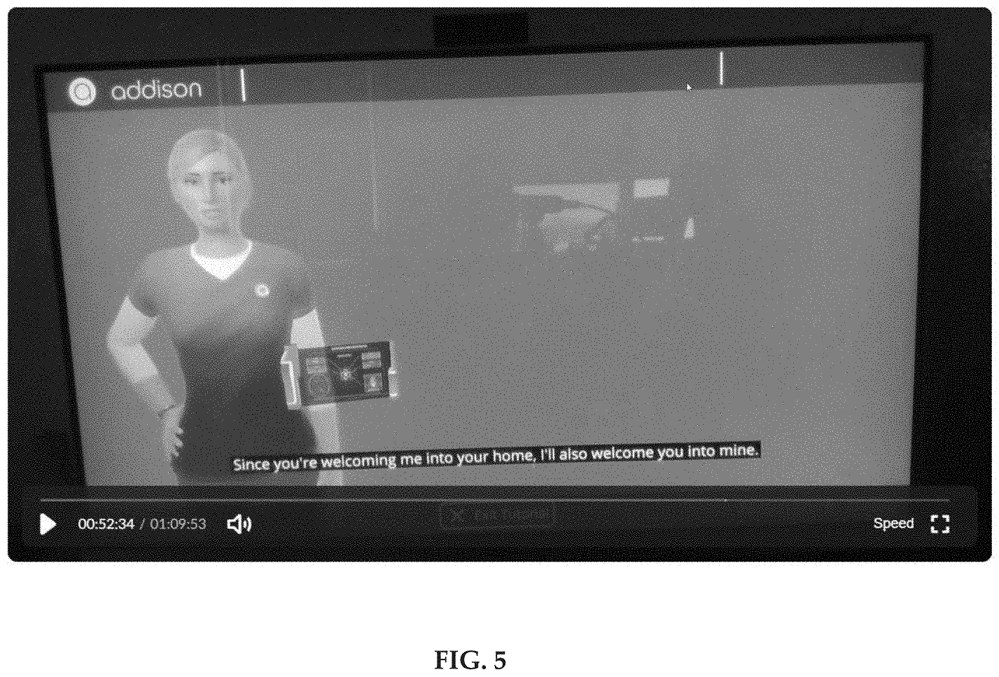

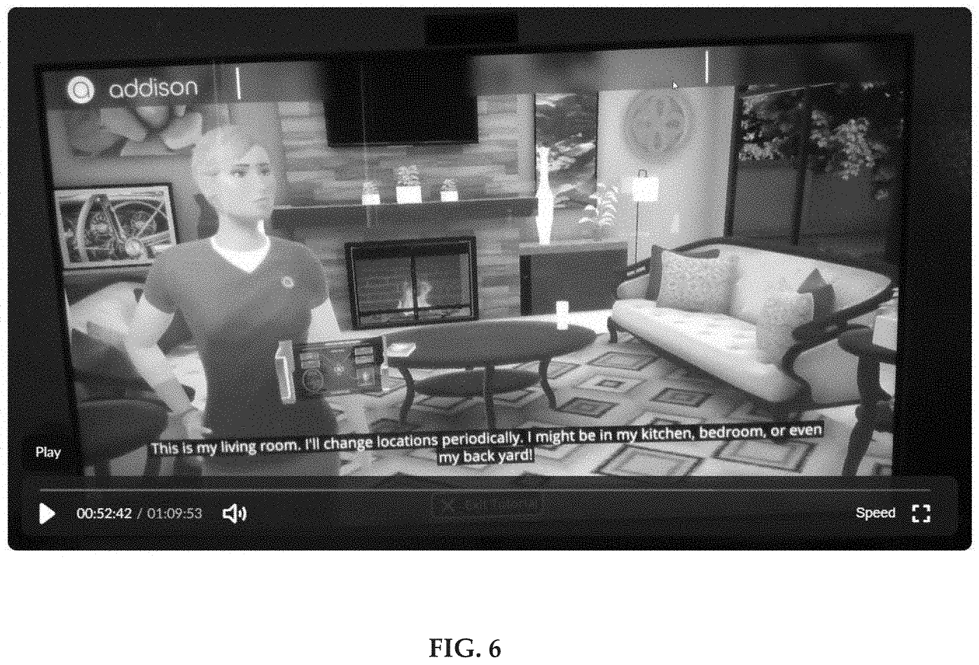

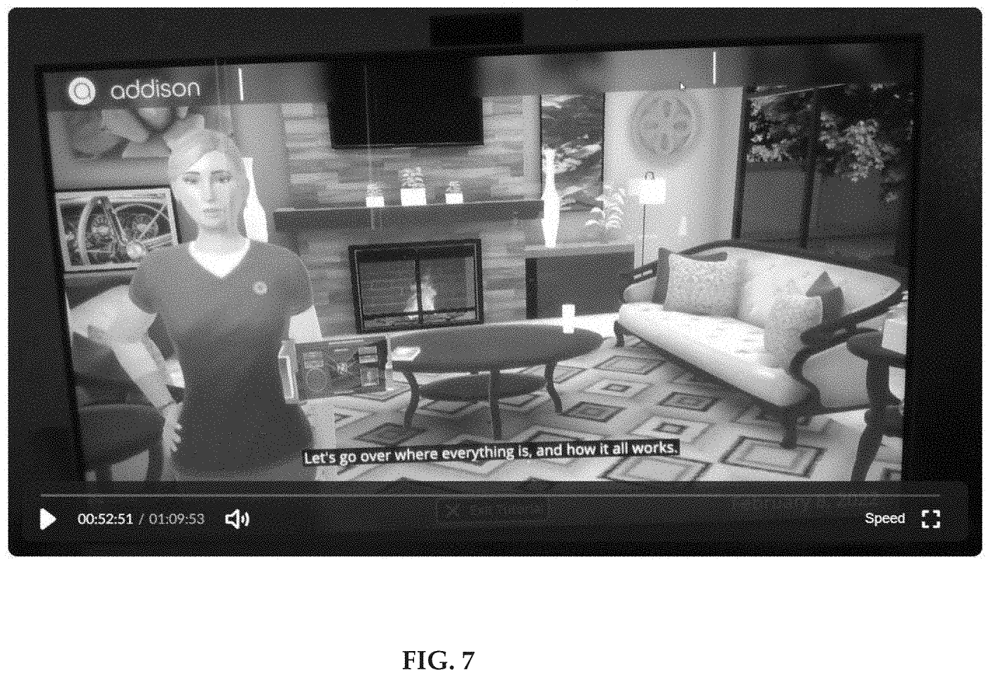

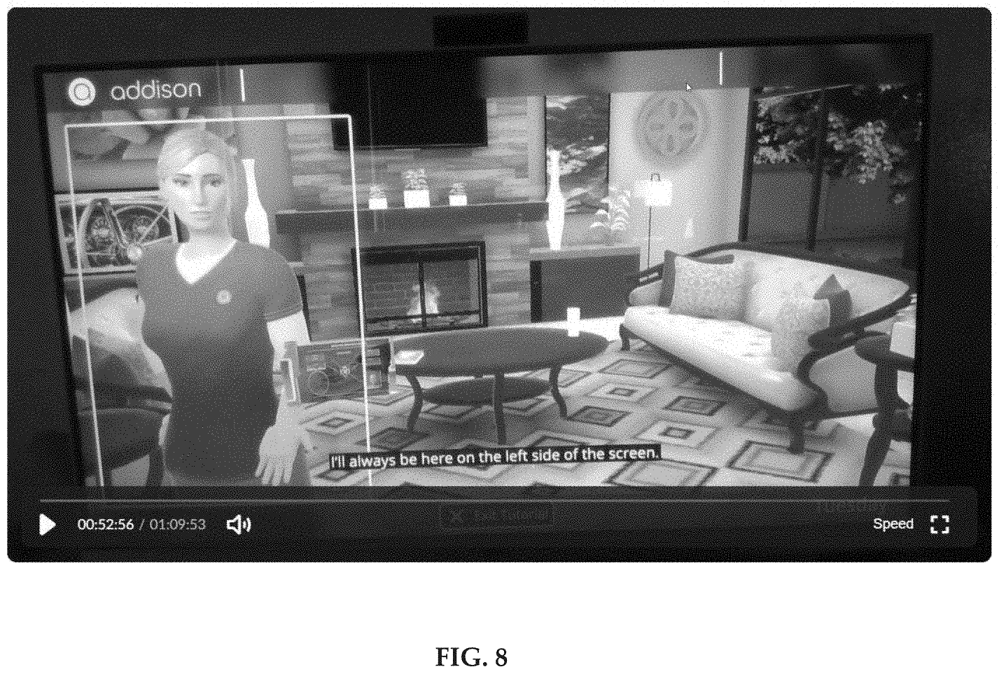

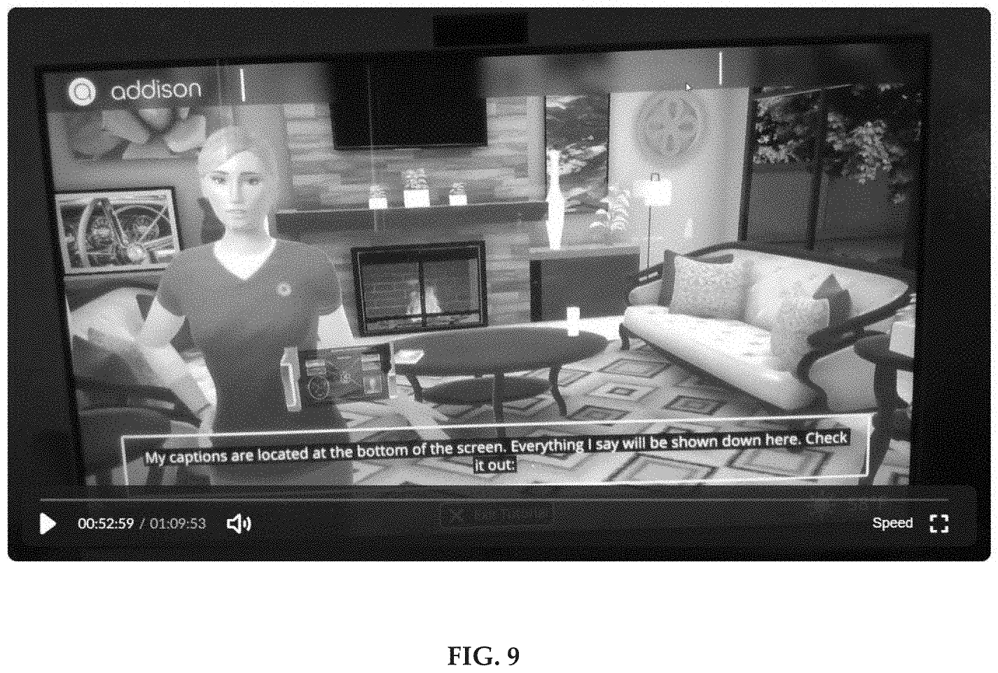

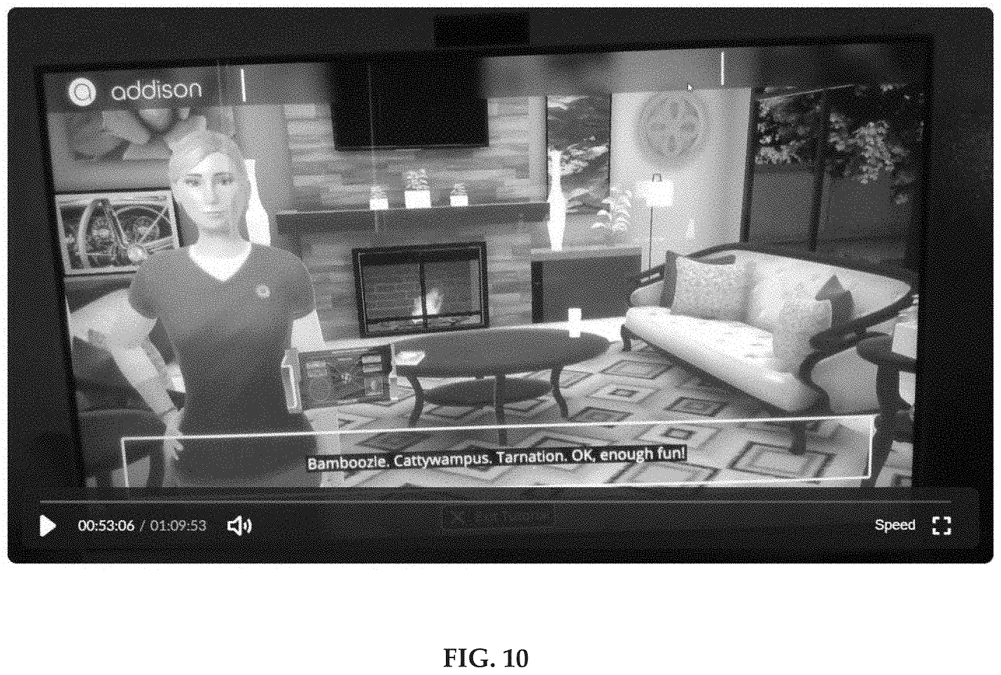

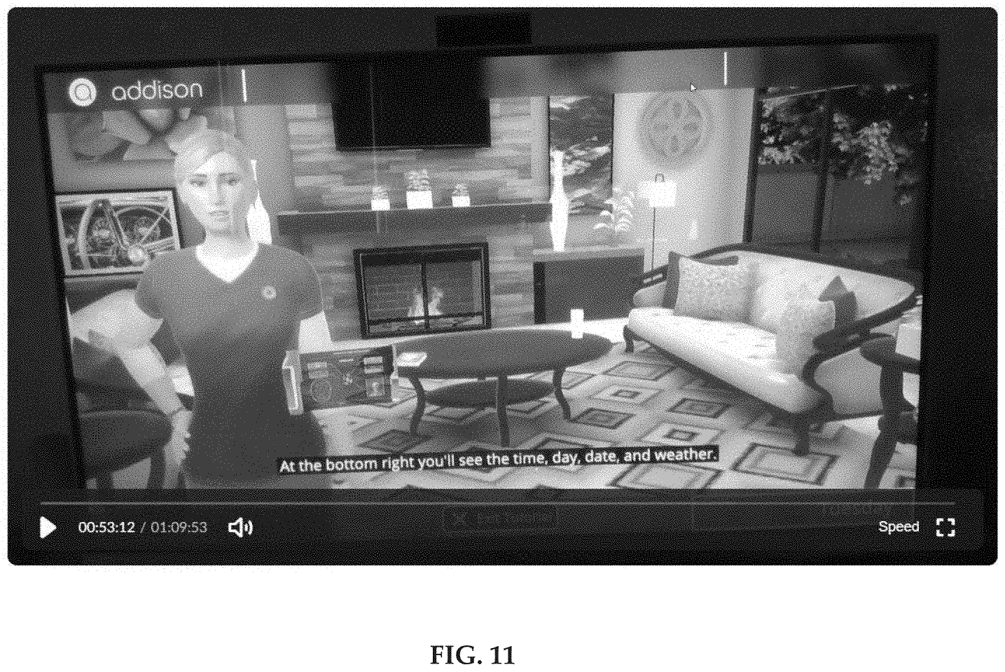

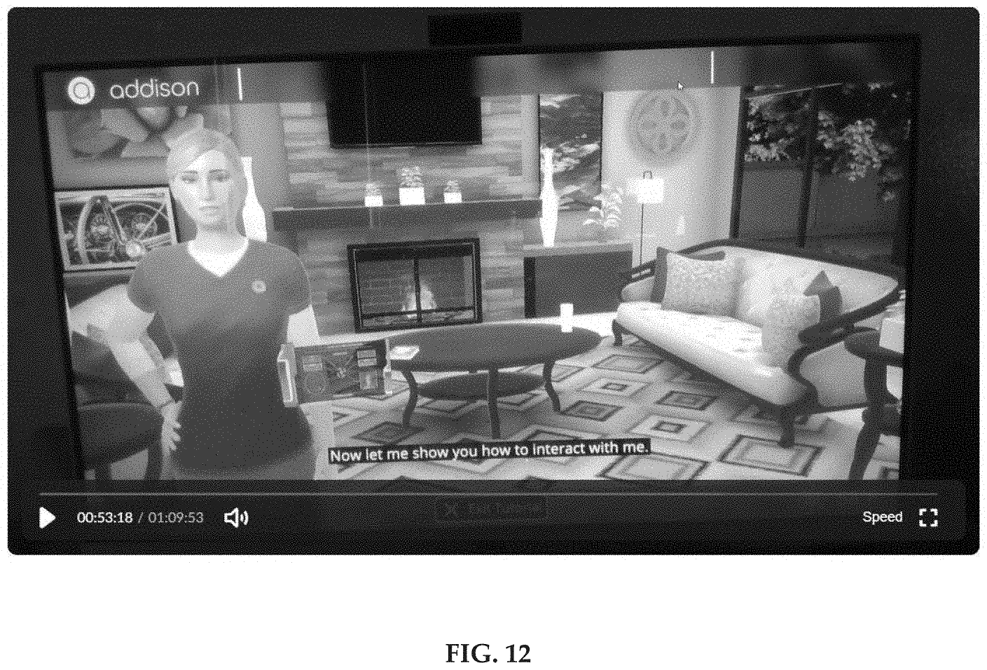

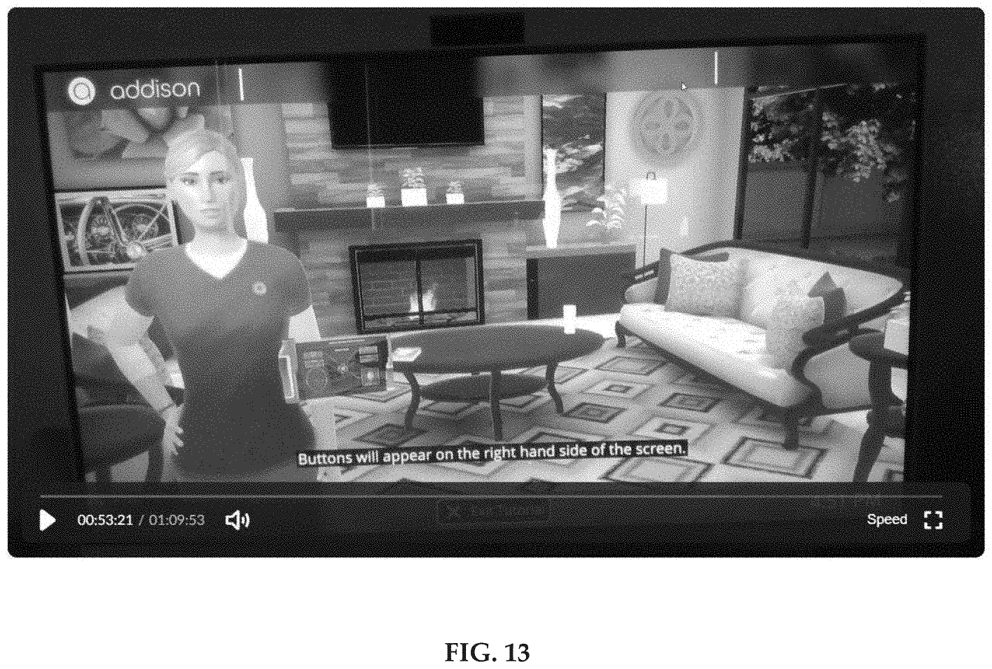

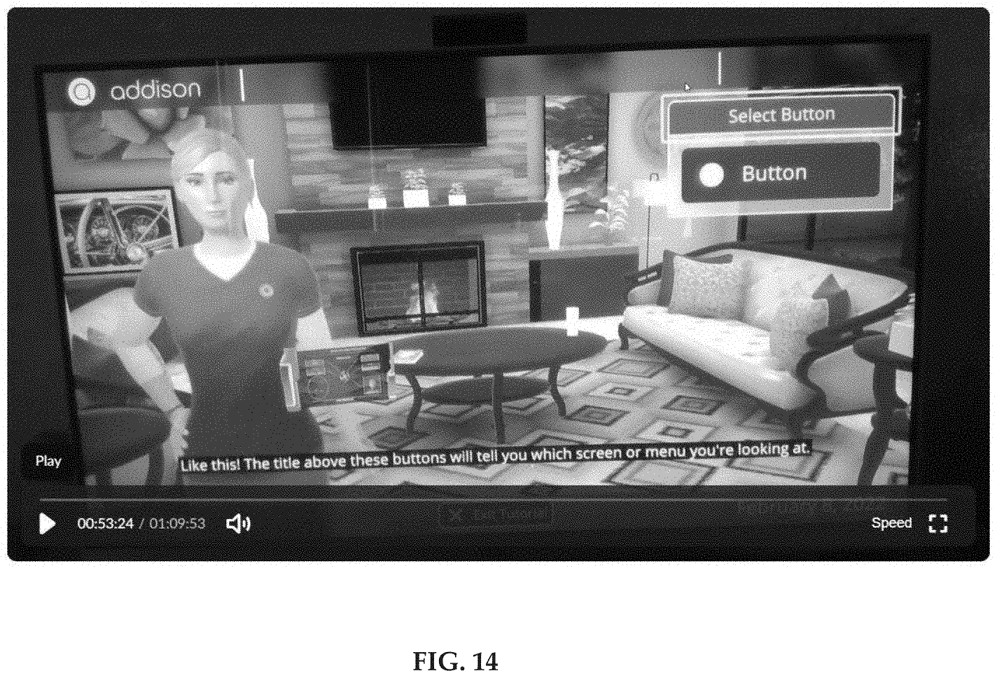

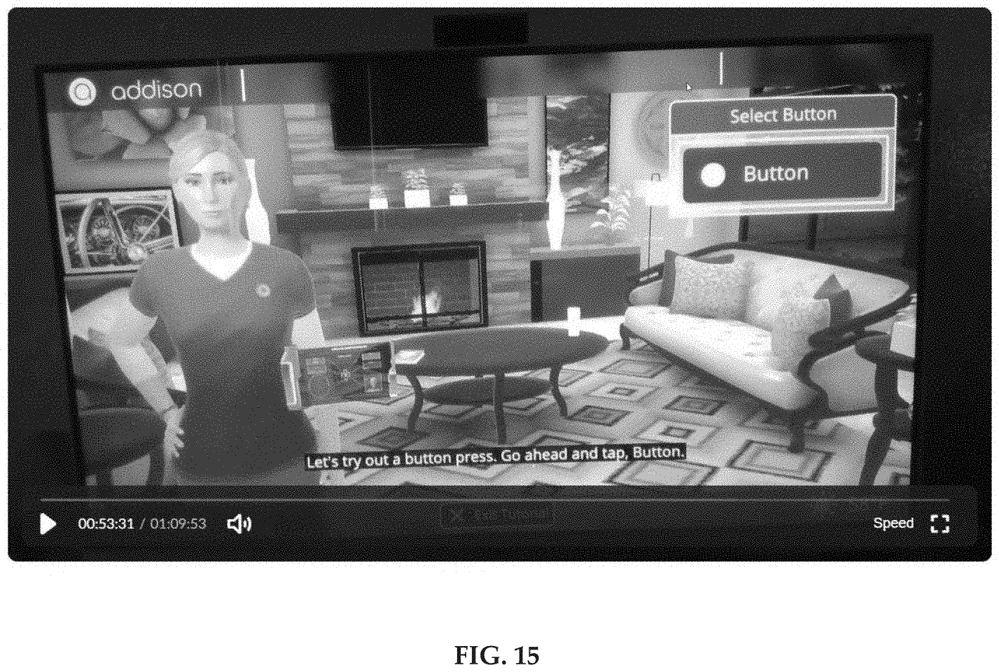

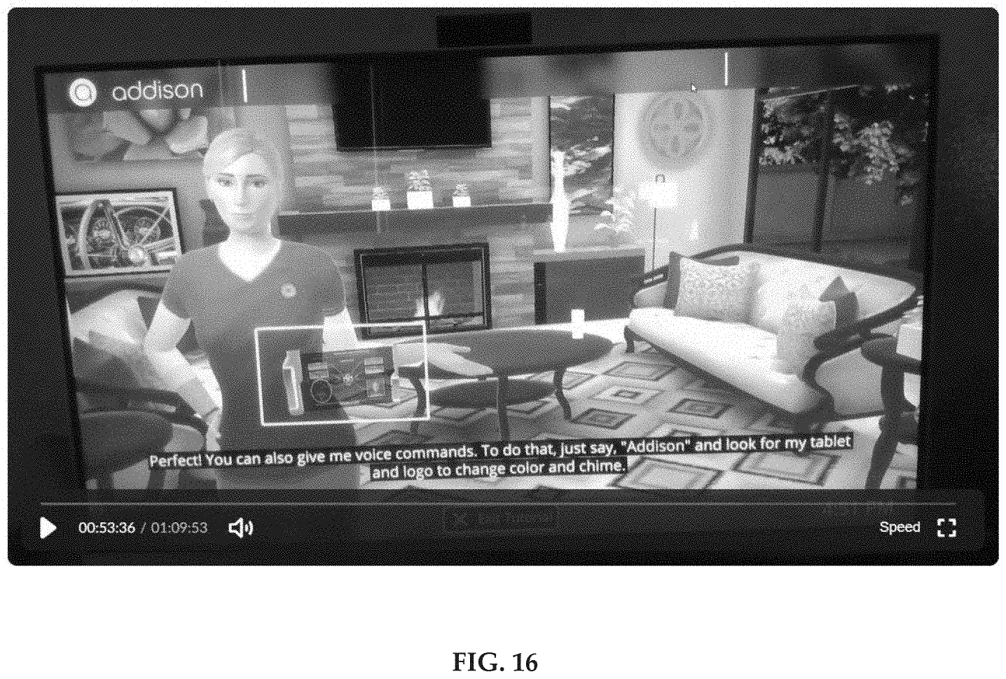

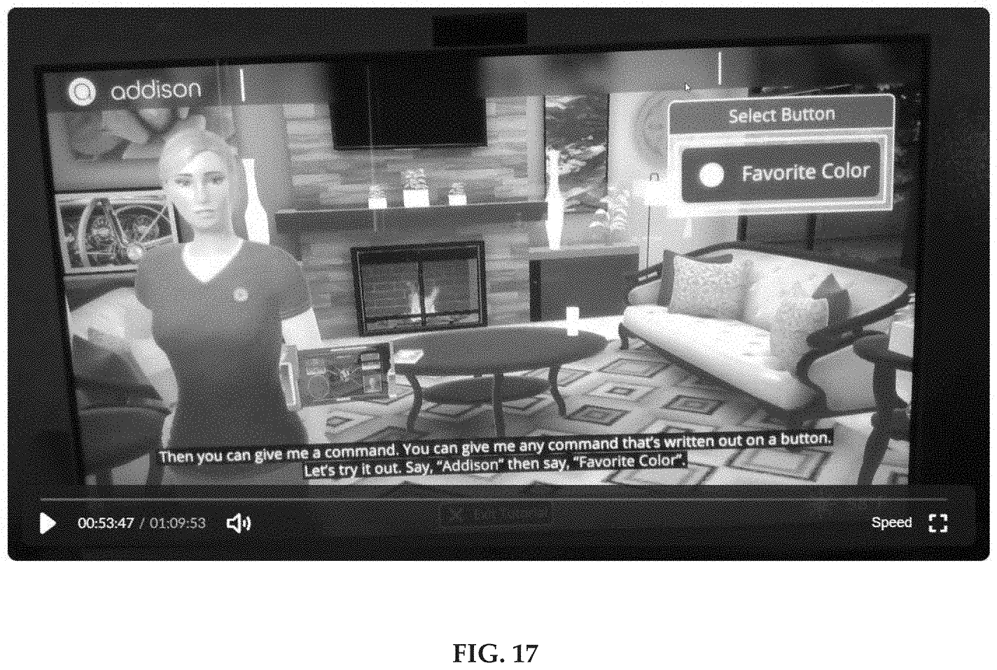

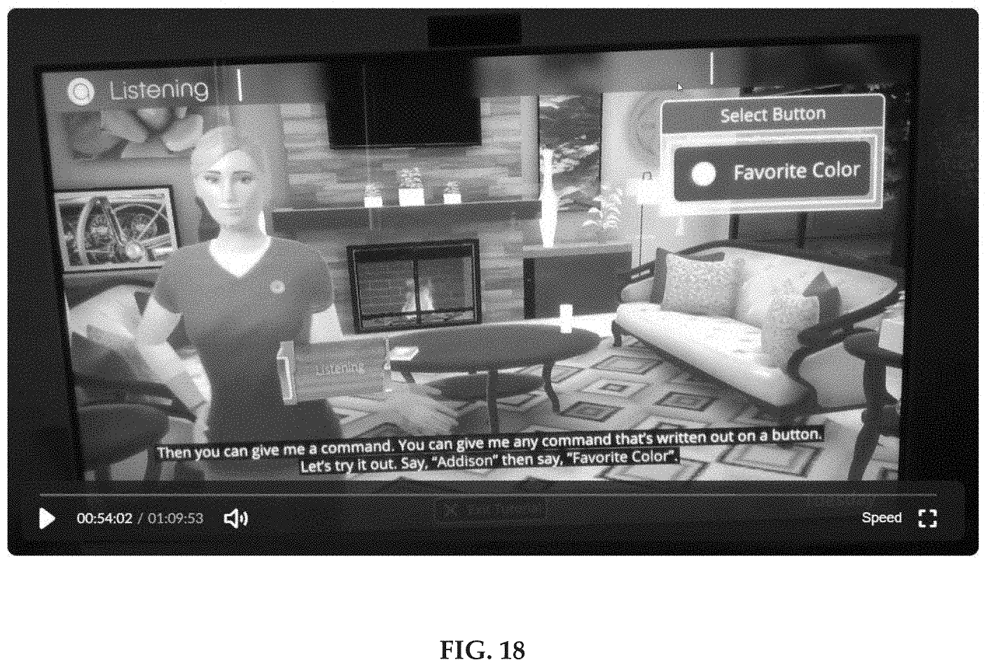

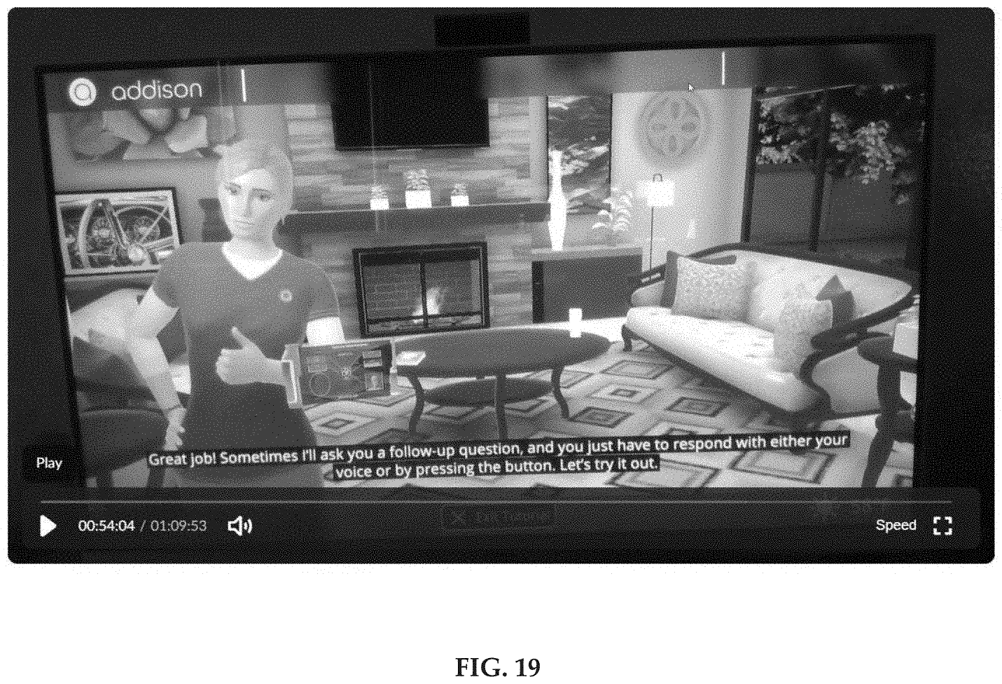

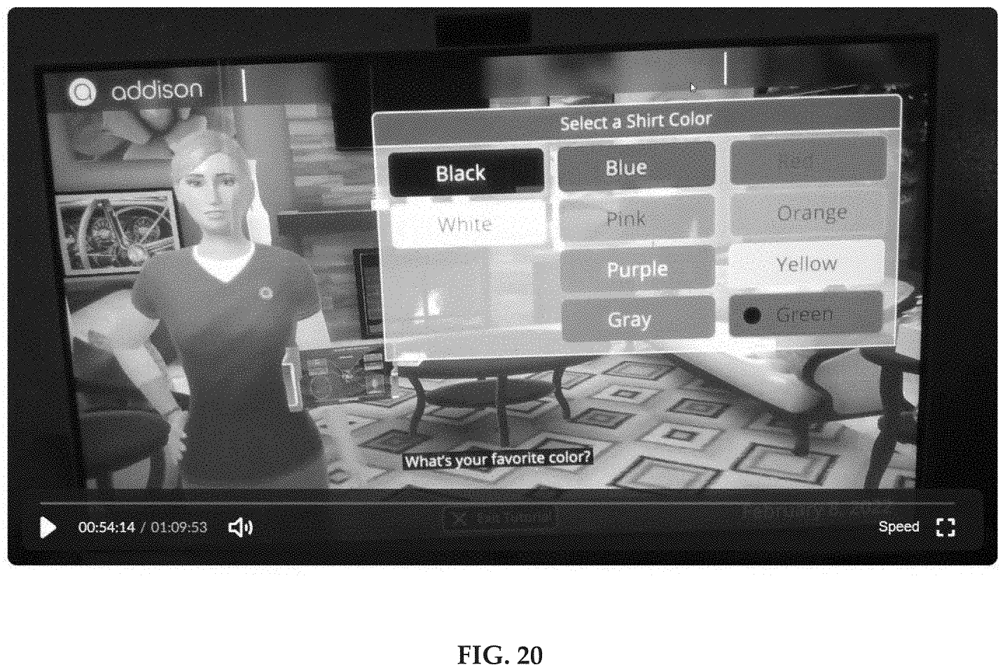

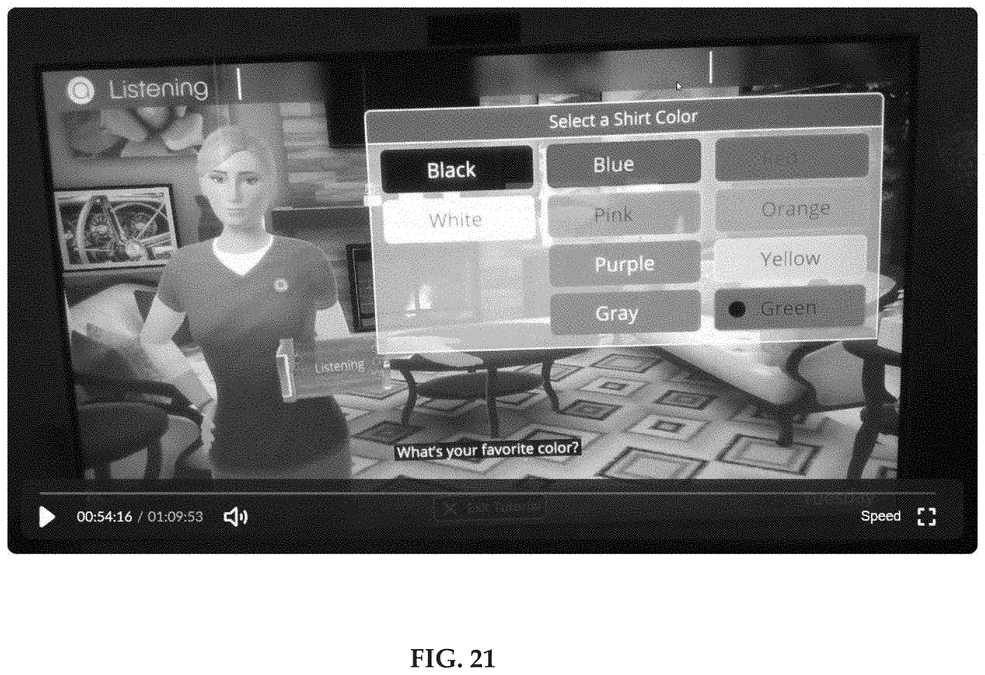

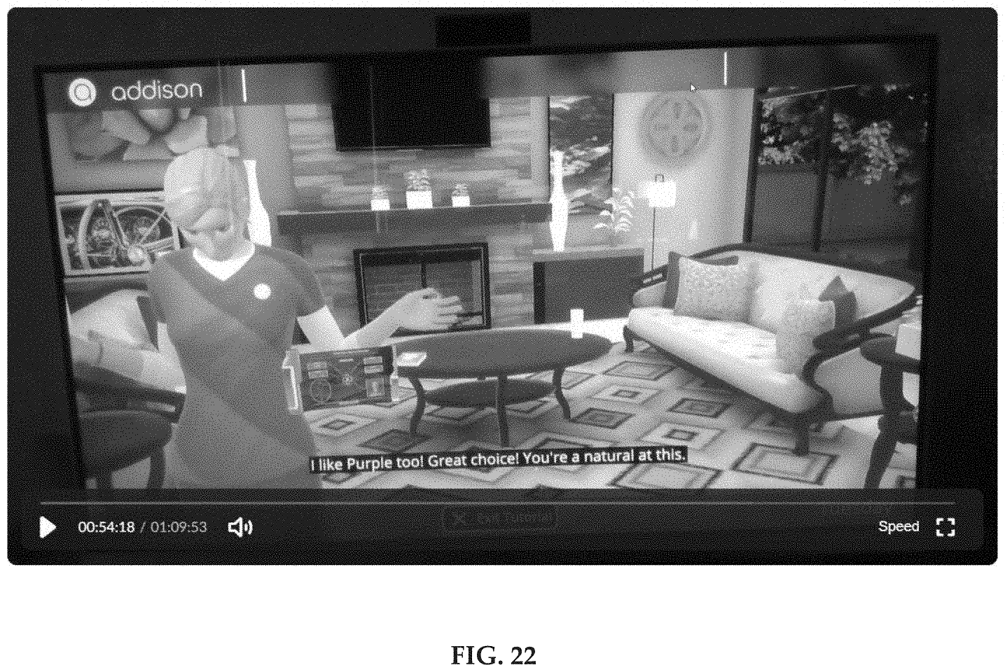

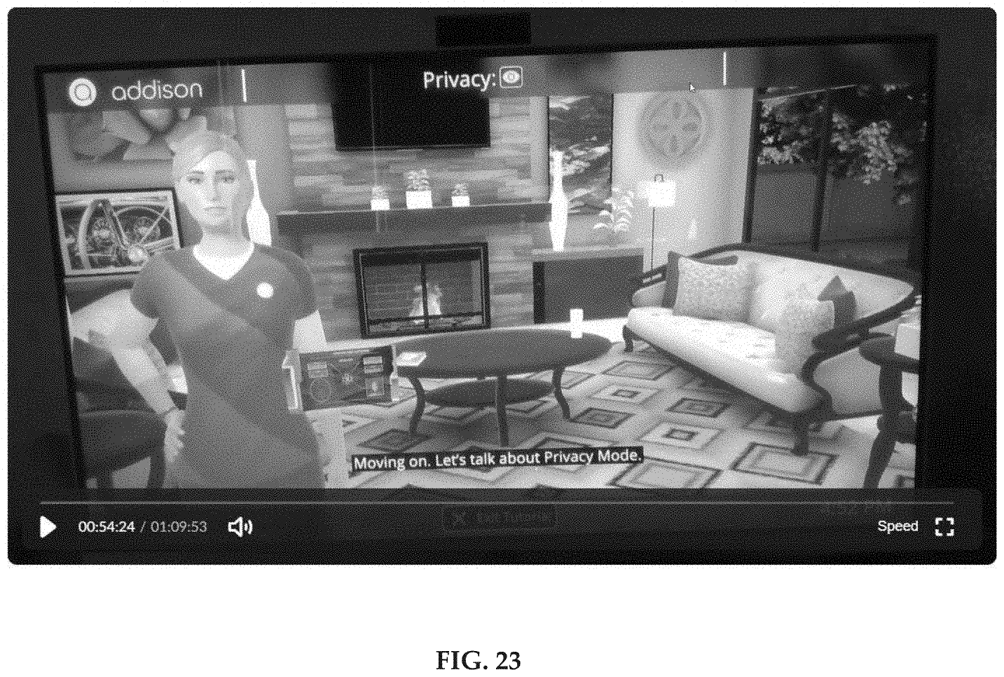

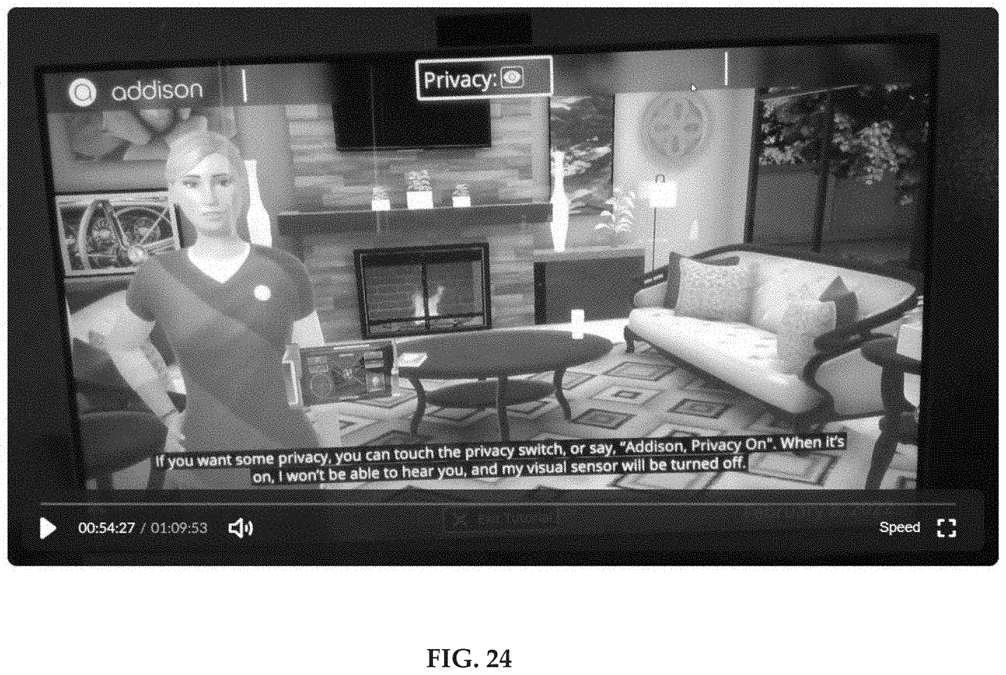

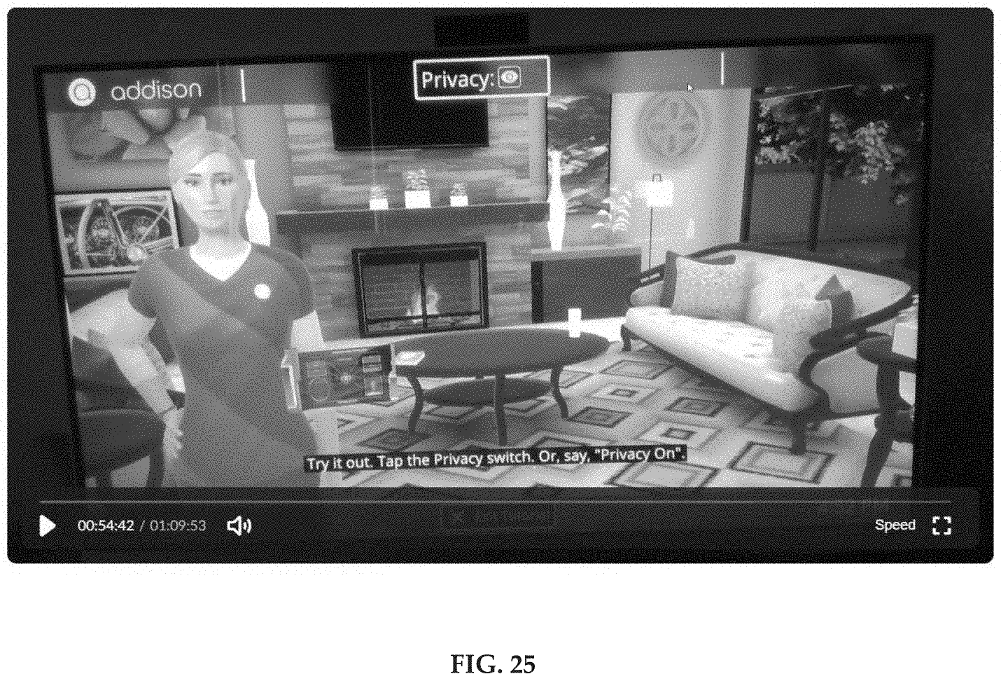

FIG. 1 is a schematic diagram of an example secure health messaging system (hereinafter system 100) for practicing aspects of the present disclosure. The system 100 comprises a data retention system 102, a health analytics system 104, a web services layer 106, and an application server layer 108 that provides, for example, modeling. Some or all of the activities occur over one or more network/communication links 118.

In some embodiments, the data retention system 102 and the health analytics system 104 are in secure isolation from a remainder of the secure messaging system 100 through a security protocol or layer. The data retention system 102 can also provide additional services such as logic, data analysis, risk model analysis, security, data privacy controls, data access controls, disaster recovery for data and web services—just to name a few.

The web services layer 106 generally provides access to the data retention system 102. According to some embodiments, the application server layer 108 is configured to provide a user-facing unity application 110 that accesses the data retention 102 and the health analytics 104 systems through the web services layer 106.

In one or more embodiments, the application server layer 108 performs asynchronous processing based on user interaction with a health messaging application that processes data from a user via the user-facing unity application 110. A health messaging application can reside and execute on the application server layer 108. In other embodiments, the health messaging application may reside with the health analytics system 104. In another embodiment, the health messaging application can be a client-side, downloadable application. Networkable health care devices 112, according to exemplary embodiments, may include a blood pressure monitor, glucometer, pro health hub, pulse oximeter and/or a thermometer. These devices may transmit information over a network, such as the Internet, to the system 100.

The systems of the present disclosure may implement security features that involve the use of multiple security tokens to provide security in the system 100. Security tokens are used between the web services layer 106 and application server layer 108.

In some embodiments, the system 100 implements an architected message bus 114. Rather than performing the refresh, which could involve data intensive and/or compute or operational intensive procedures by the system 100, the message bus 114 allows the request for refresh to be processed asynchronously by a batching process and provides a means for allowing the user-facing unity application to provide a view to the user, allowing the user to continue to access data without waiting on the system 100 to complete its refresh.

Also, latency can be remediated at the user-facing unity application 110 based on the manner with which the user-facing unity application 110 is created and how the data that is displayed through the user-facing unity application 110 is stored and updated. For example, data displayed on the user-facing unity application 110 that changes frequently can cause frequent and unwanted refreshing of the entire user-facing application and interactive graphical user interfaces (“GUIs”). The present disclosure provides a solution to this issue by separating what is displayed on the GUI with the actual underlying data. The underlying data displayed on the GUI of the user-facing unity application 110 can be updated, as needed, on a segment-by-segment basis (could be defined as a zone of pixels on the display) at a granular level, rather than updating the entire GUI. That is, the GUI that renders the underlying data is programmatically separate from the underlying data cached by the client (e.g., device rendering the GUIs of the user-facing unity application 110). Due to this separation, when data being displayed on the GUI changes, re-rendering of the data is performed at a granular level, rather than at the page level. This process represents another example solution that remedies latency and improves user experiences with the user-facing unity application 110.

To facilitate these features, the user facing unity application 110 will listen on the message bus 114 for an acknowledgement or other confirmation that the background processes to update the user account and/or the user-facing unity application have been completed by the application server layer 108. The user-facing unity application (or even part thereof) is updated as the system 100 completes its processing. This allows the user-facing unity application 110 to be usable, but heavy lifting is being done transparently to the user by the application server layer 108. In sum, these features prevent or reduce latency issues even when an application provided through the user facing unity application 110 is “busy.” For example, a re-balance request is executed transparently by the application server layer 108 and batch engine 116. This type of transparent computing behavior by the system 100 allows for asynchronous operation (initiated from the application server layer 108 or message bus 114).

In some embodiments, a batch engine 116 is included in the system 100 and works in the background to process re-balance requests and coordinate a number of services. An example re-balance request would include an instance where a user selectively makes a data request. The batch engine 116 will transparently orchestrate the necessary operations required by the application sever layer 108 in order to obtain data.

According to some embodiments, the batch engine 116 is configured to process requests transparently to a user so that the user can continue to use the user-facing unity application 110 without disruption. For example, this transparent processing can occur when the application server layer 108 transmits a request to the web services layer 106 for data, and a time required for updating or retrieving the data meets or exceeds a threshold. For example, the threshold might specify that if the request will take more than five seconds to complete, then the batch engine 116 can process the request transparently. The selected threshold can be system configured.

In some embodiments, security of data transmission through the system 100 is improved by use of multiple security tokens. In one embodiment, a security protocol or security token utilized between the application server layer 108 and the web services layer 106.

For example, feedback responses as described herein may be transmitted back to the data retention system 102 and/or the health analytics system 104.

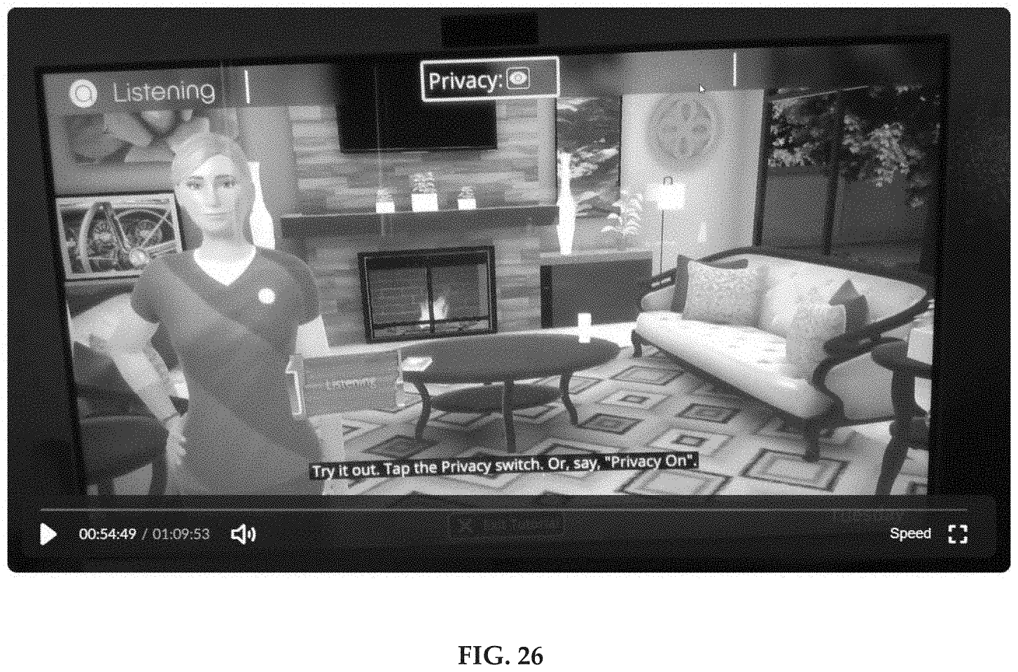

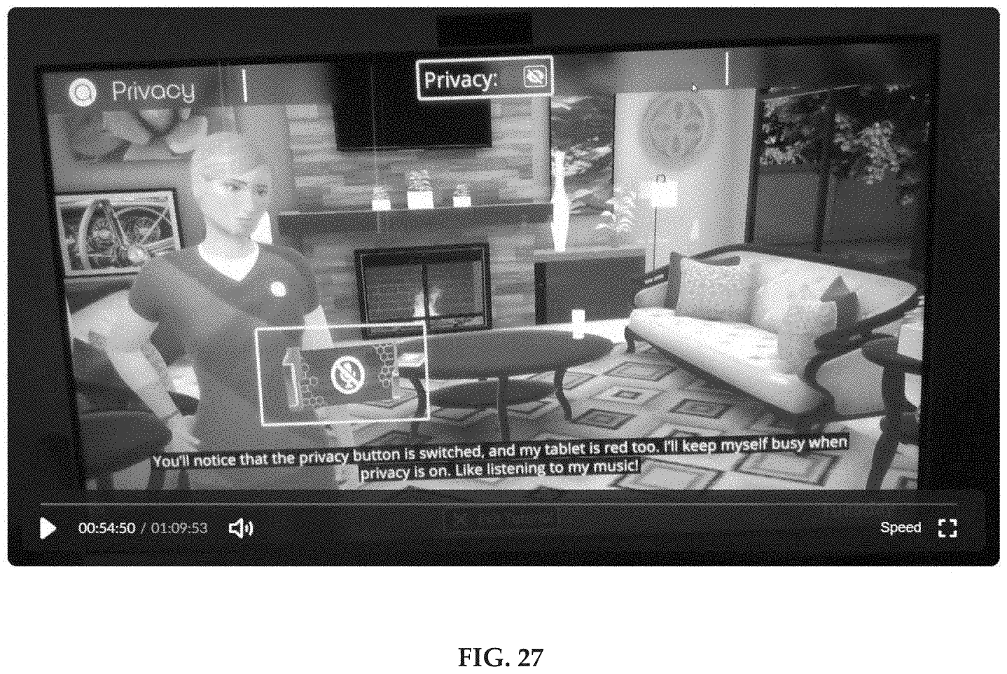

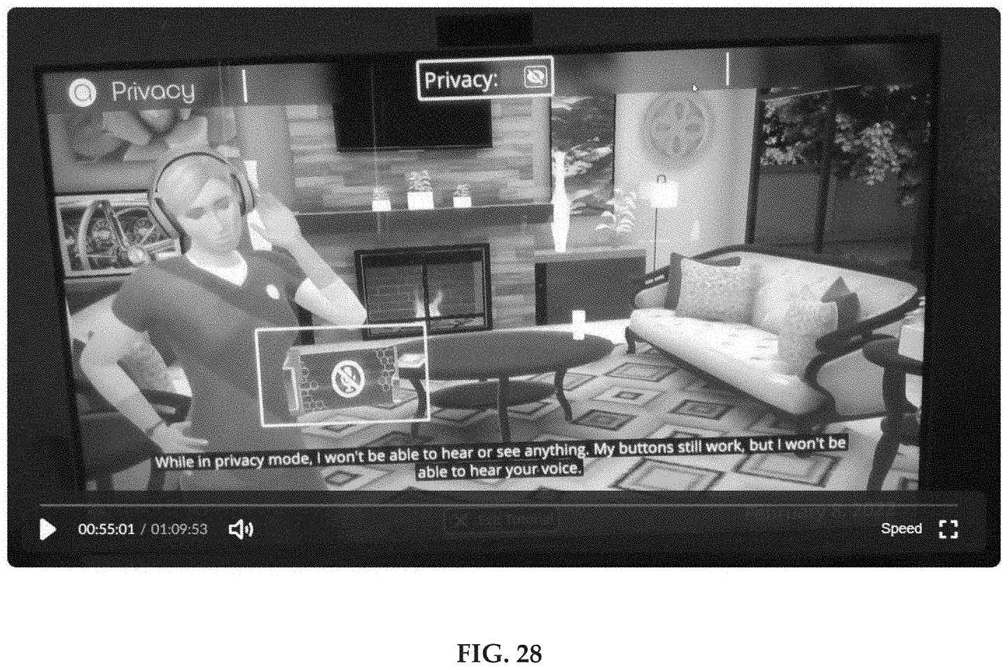

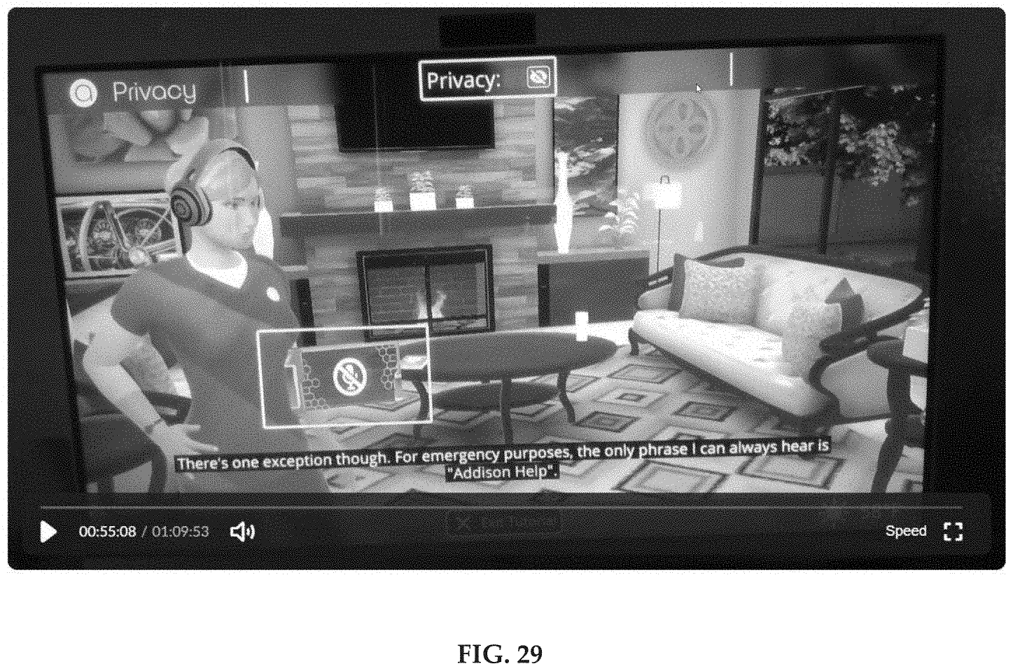

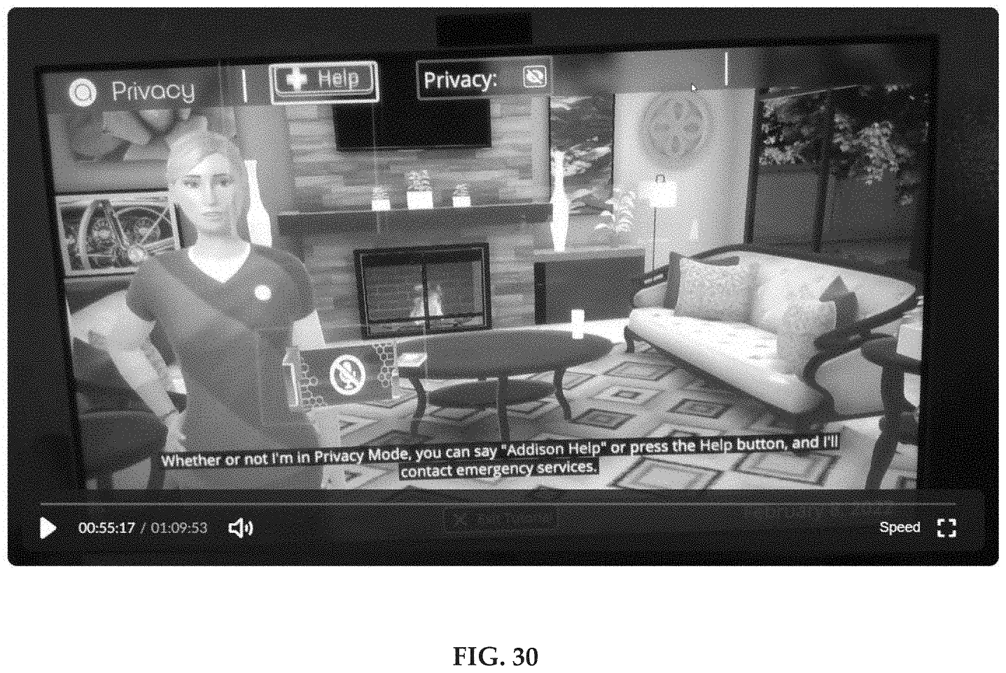

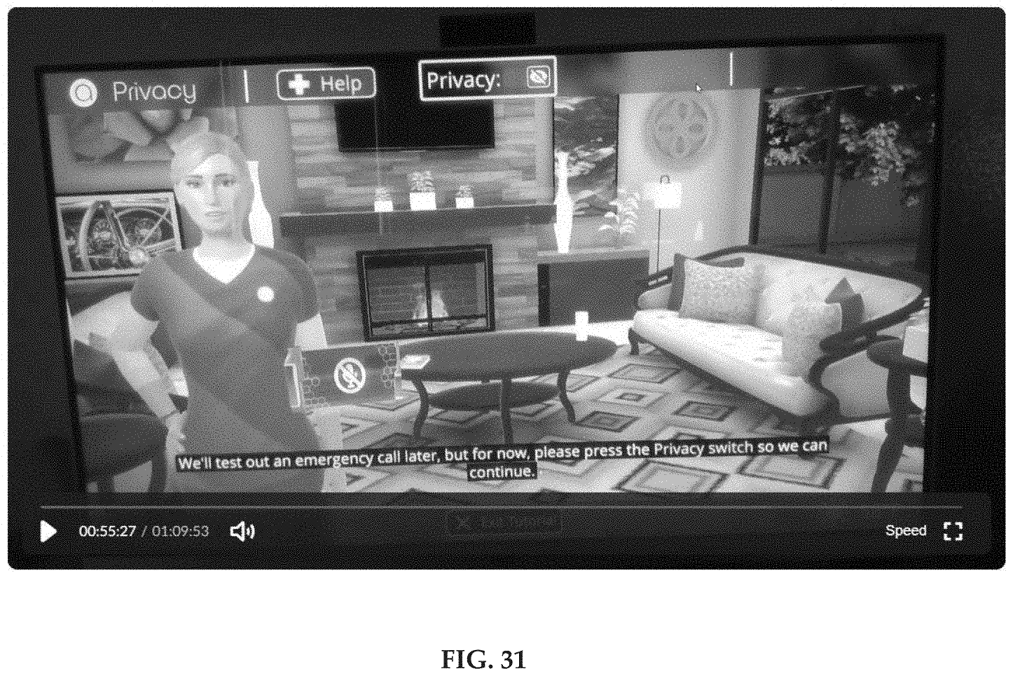

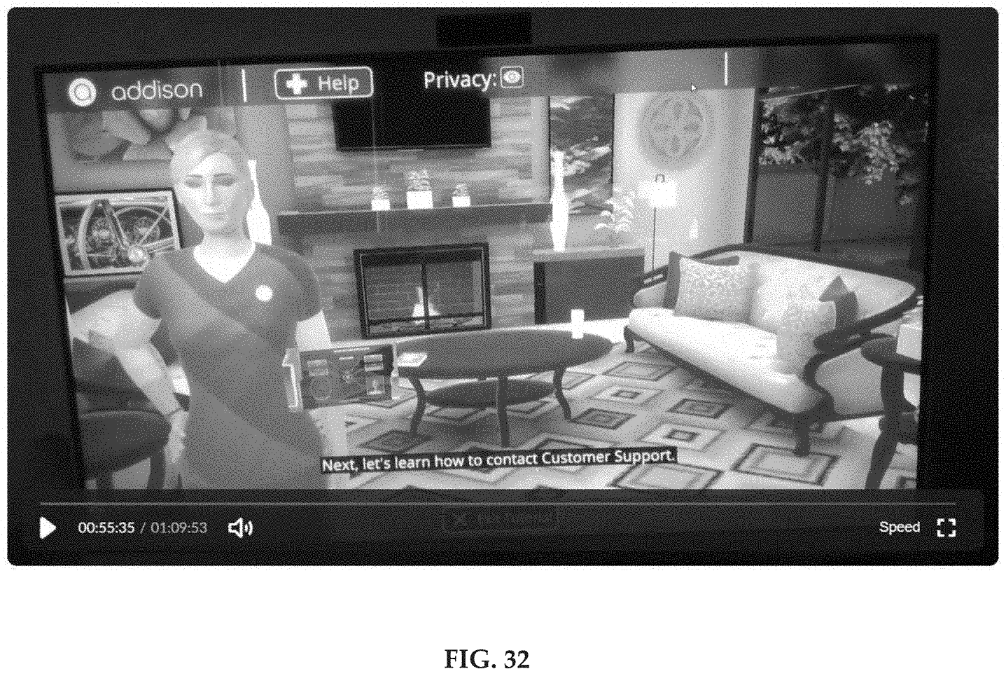

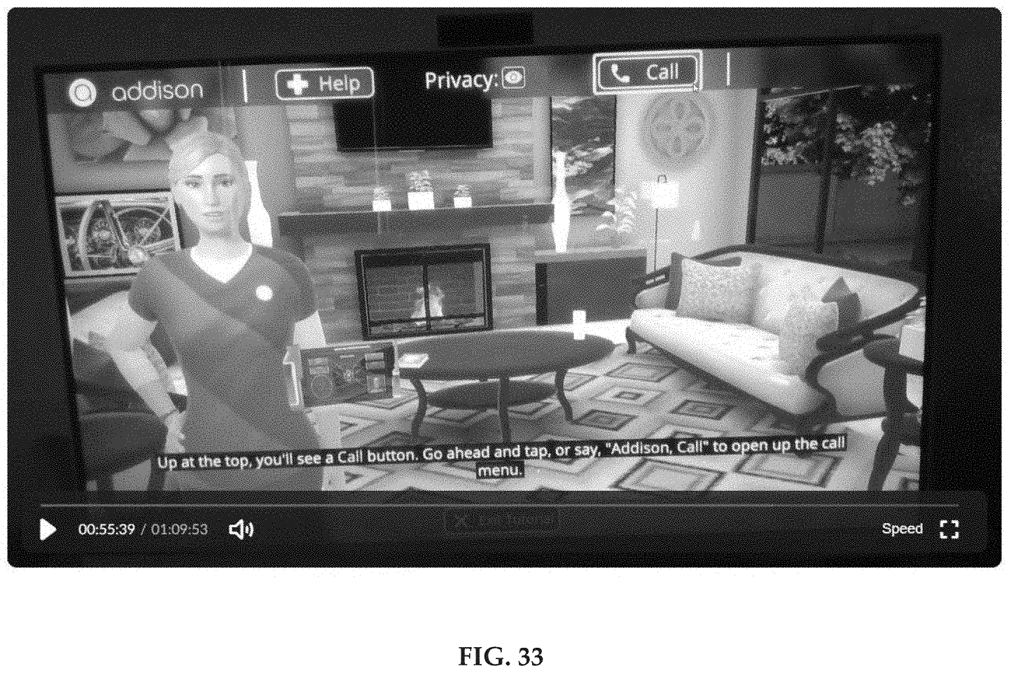

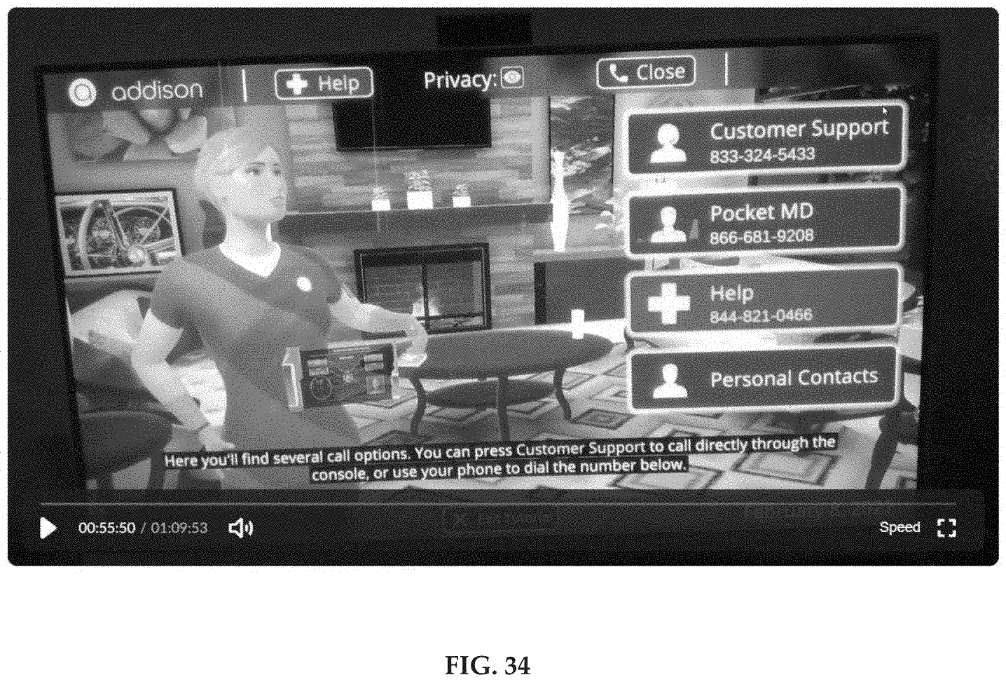

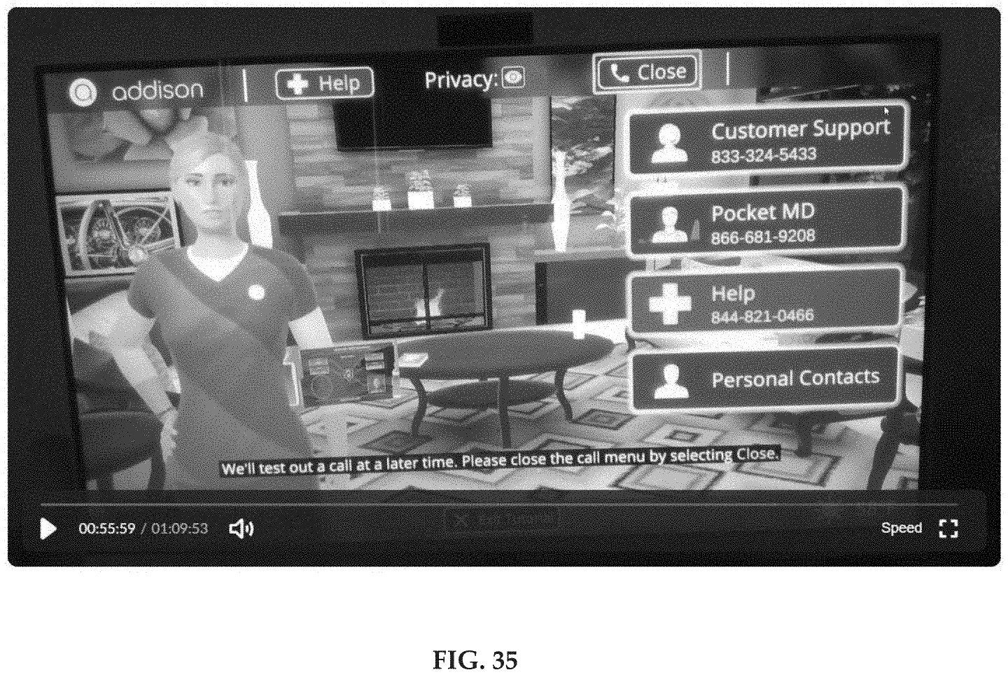

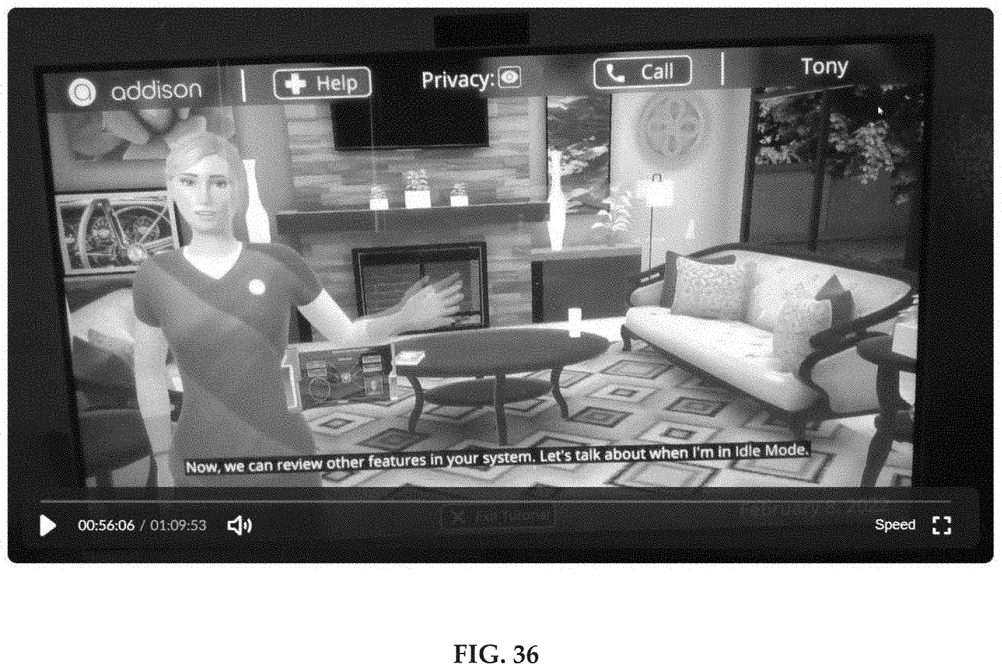

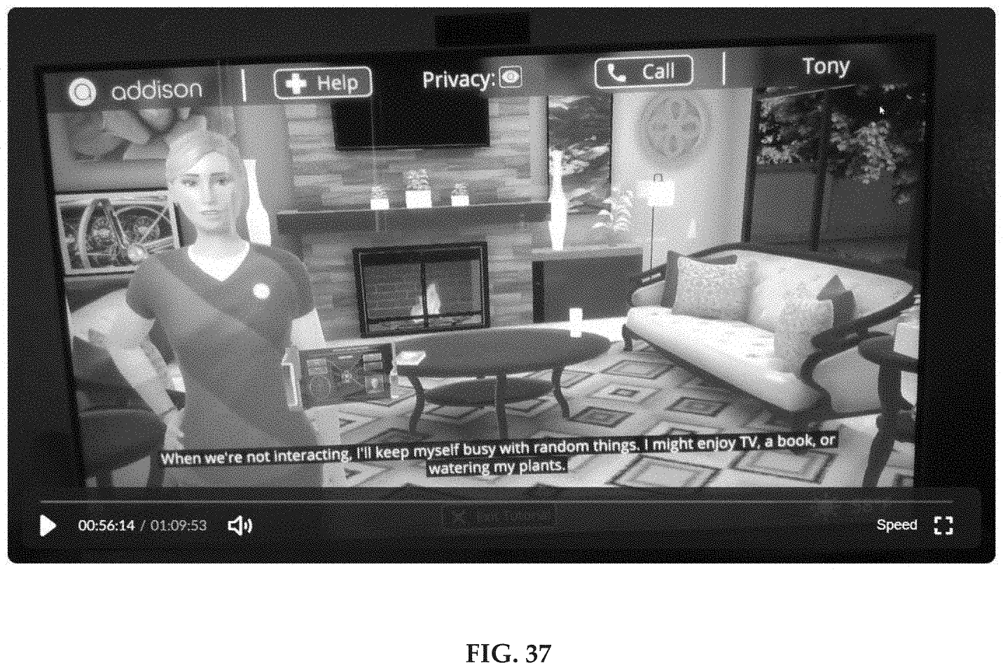

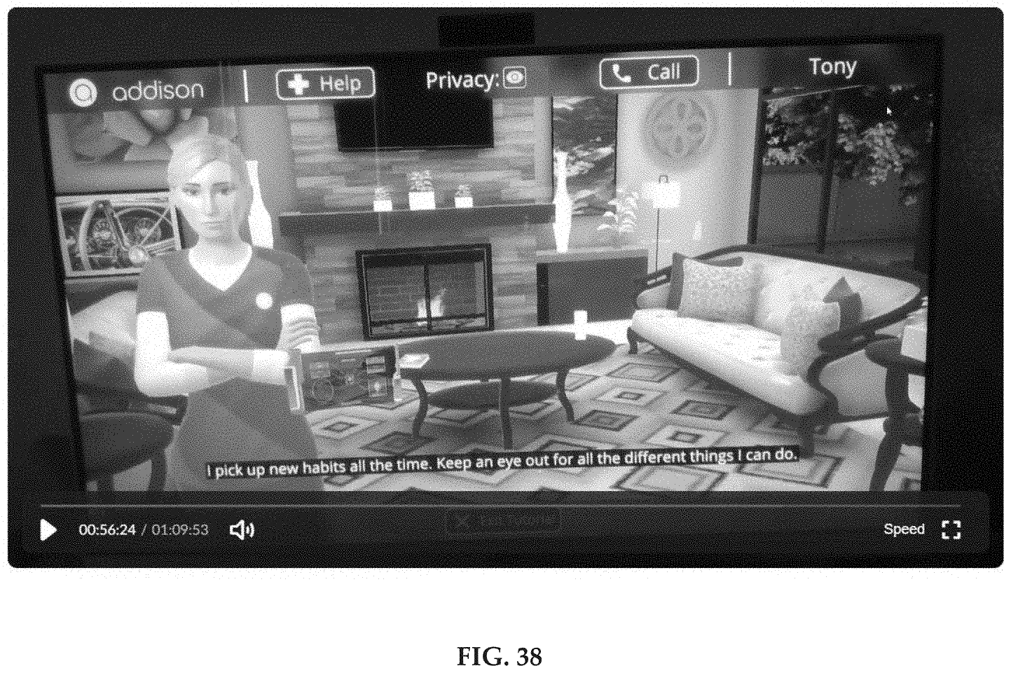

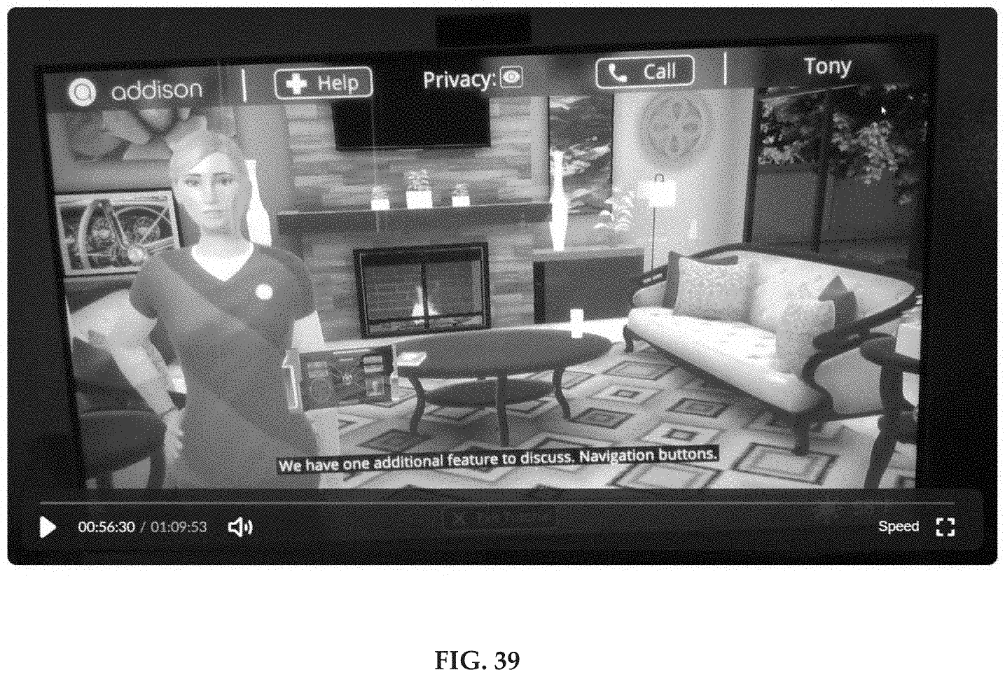

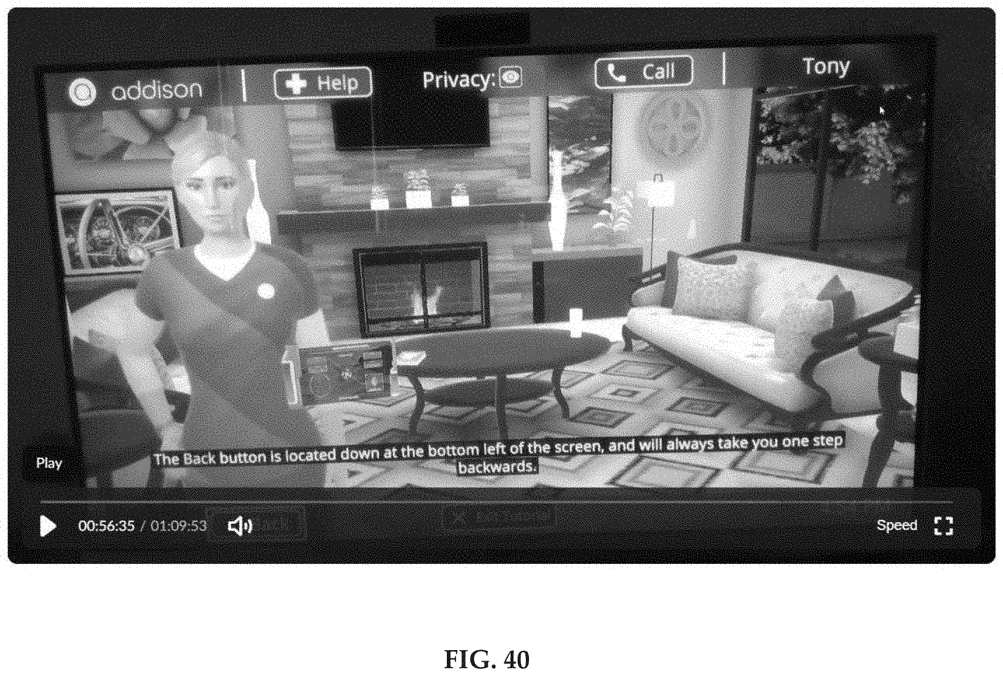

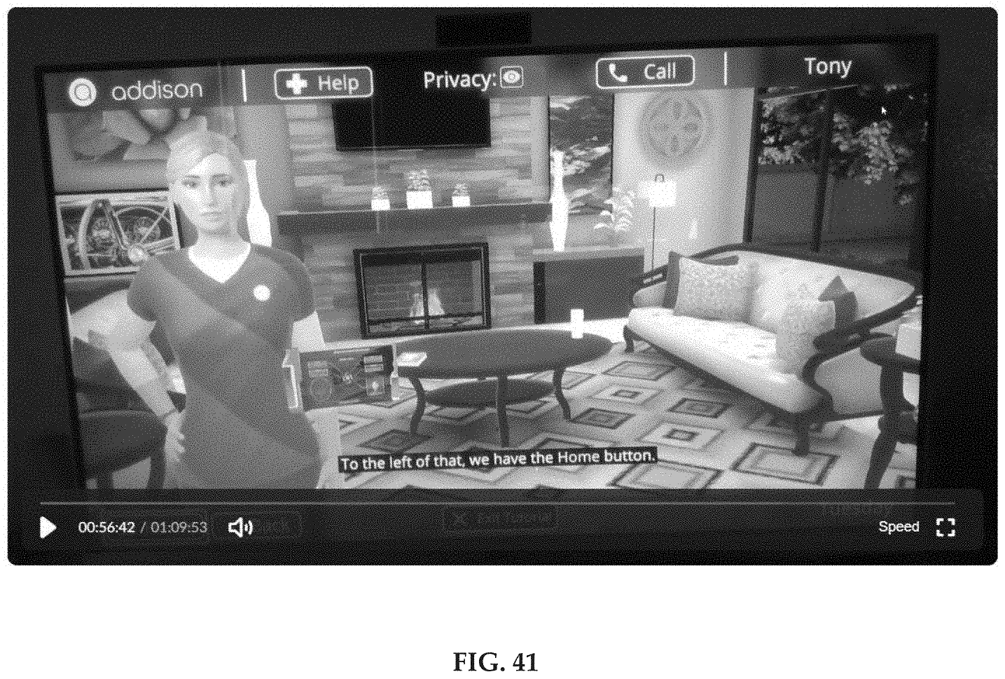

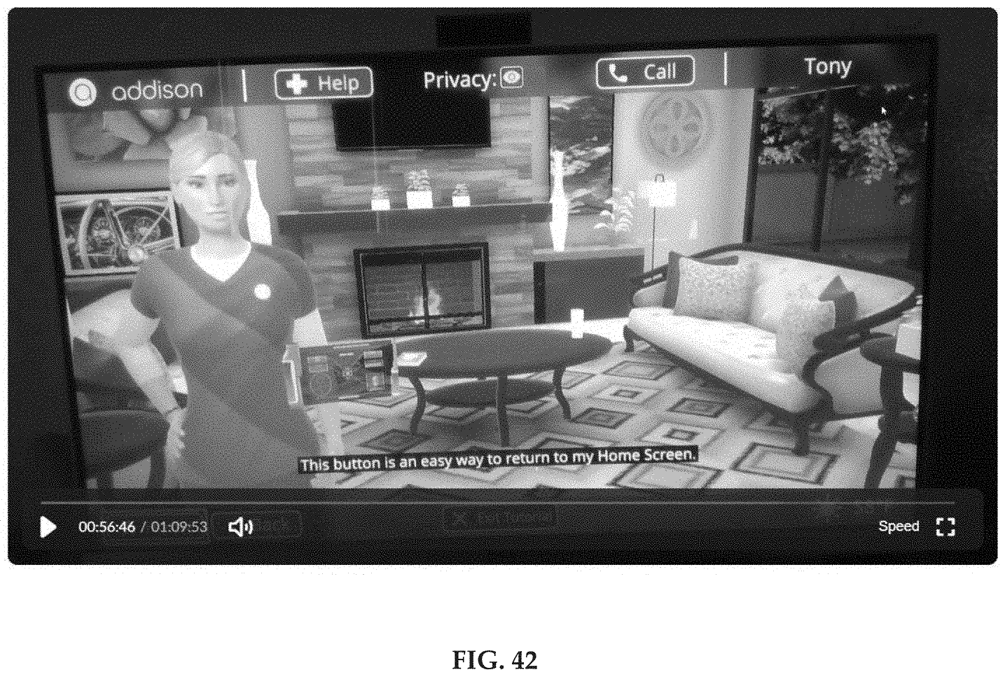

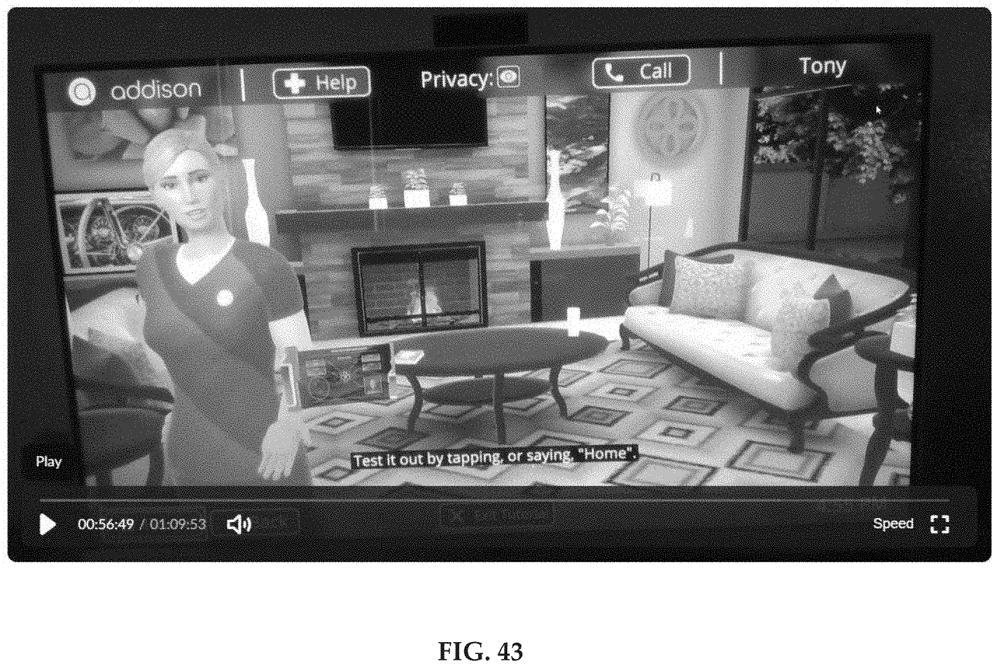

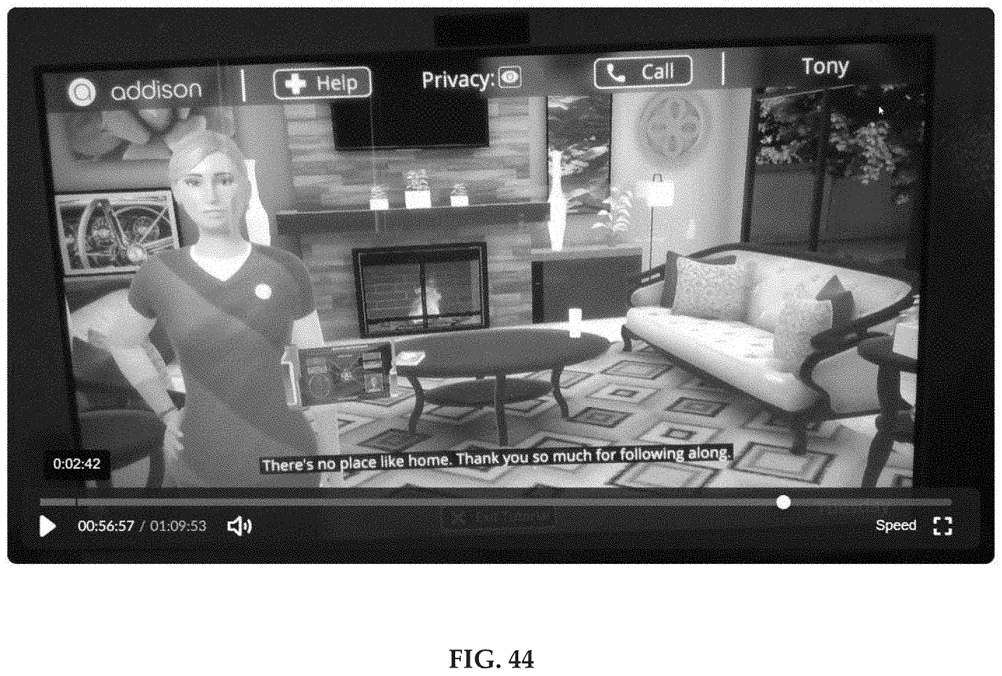

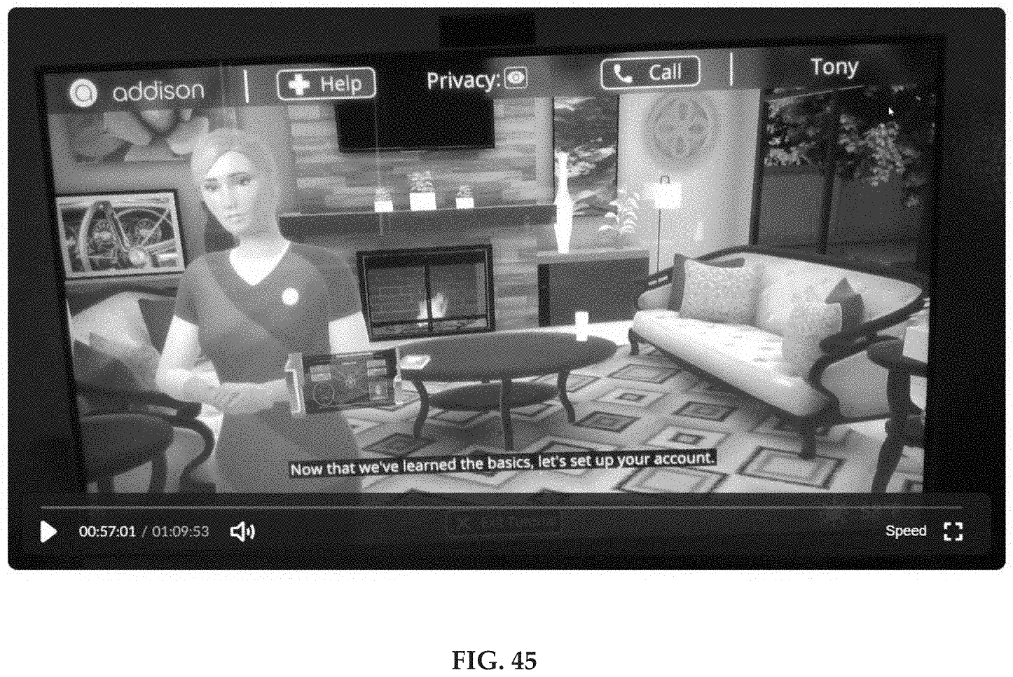

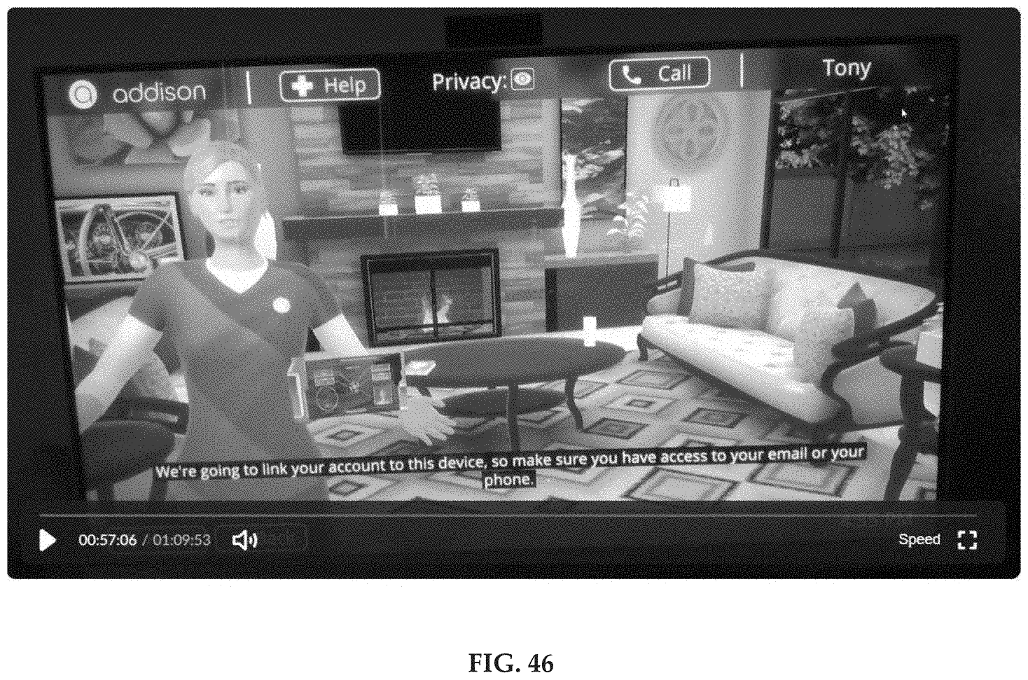

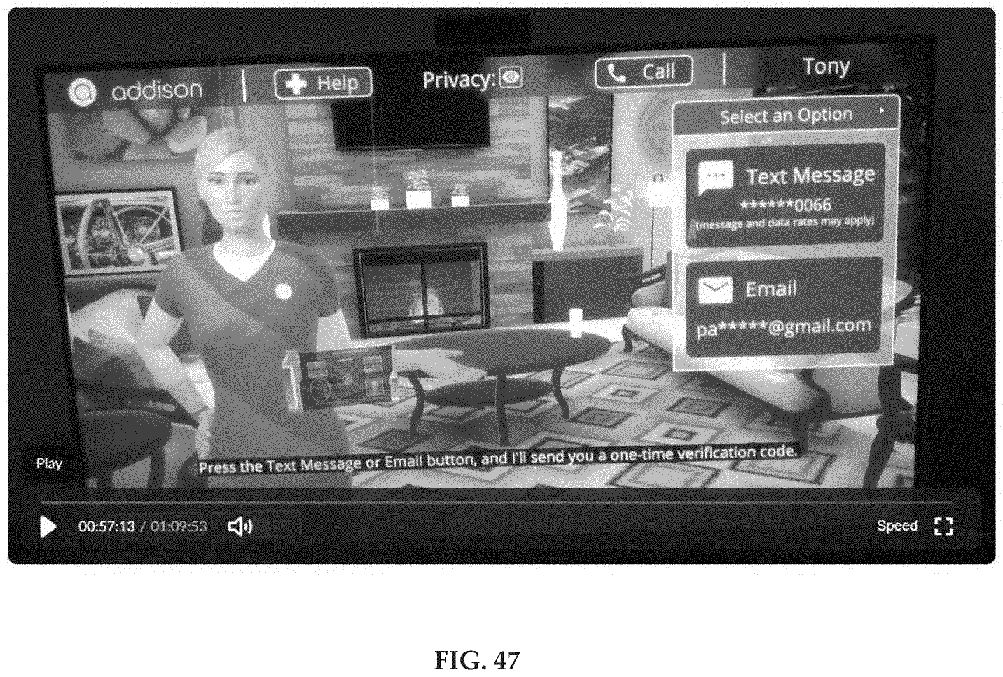

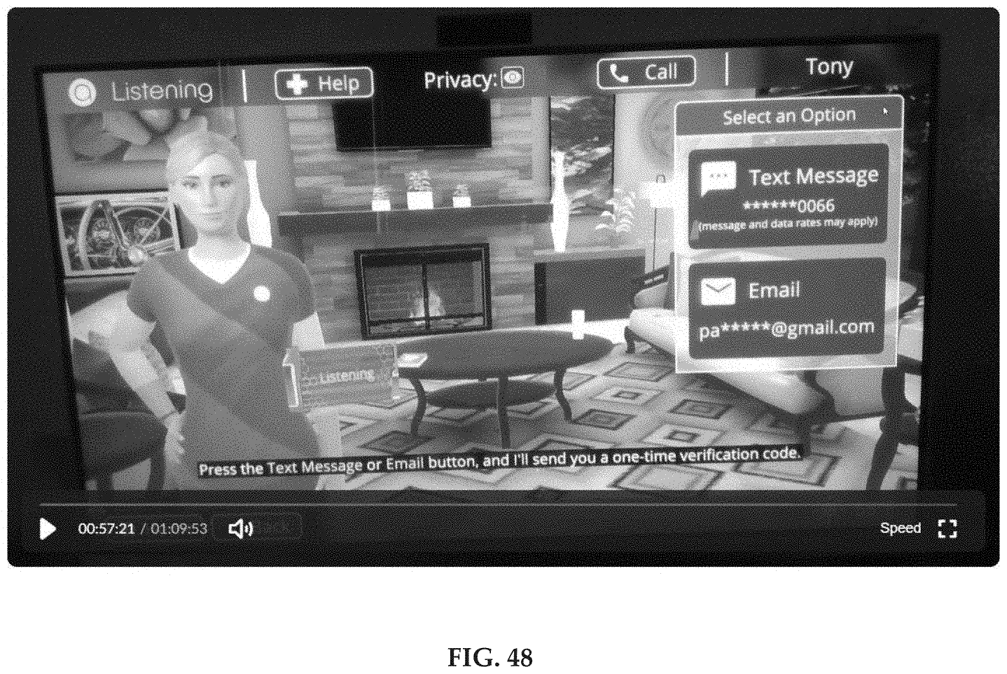

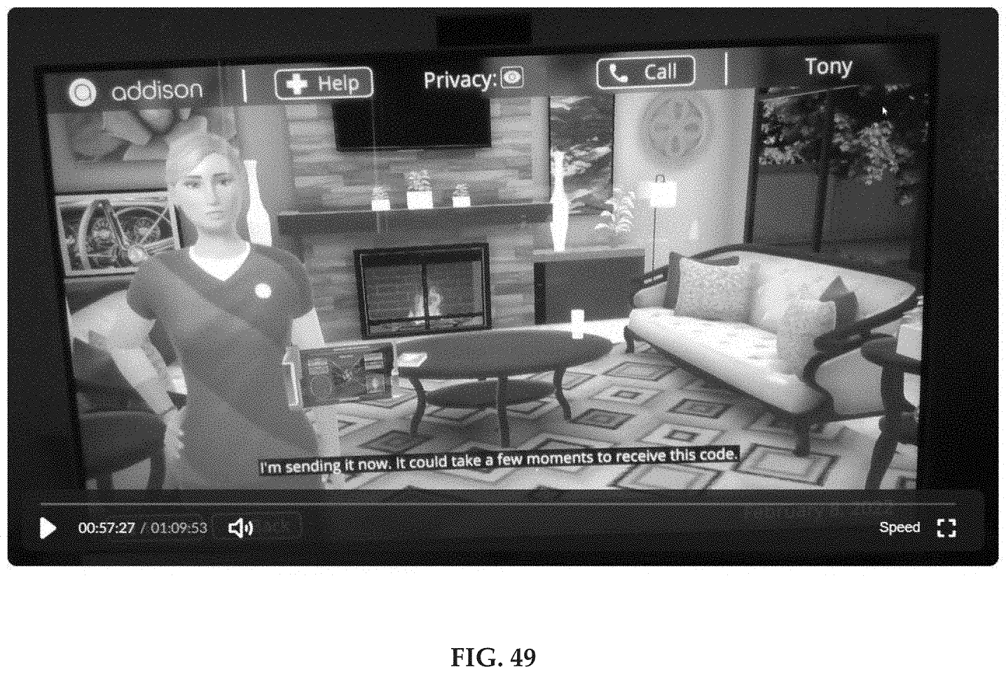

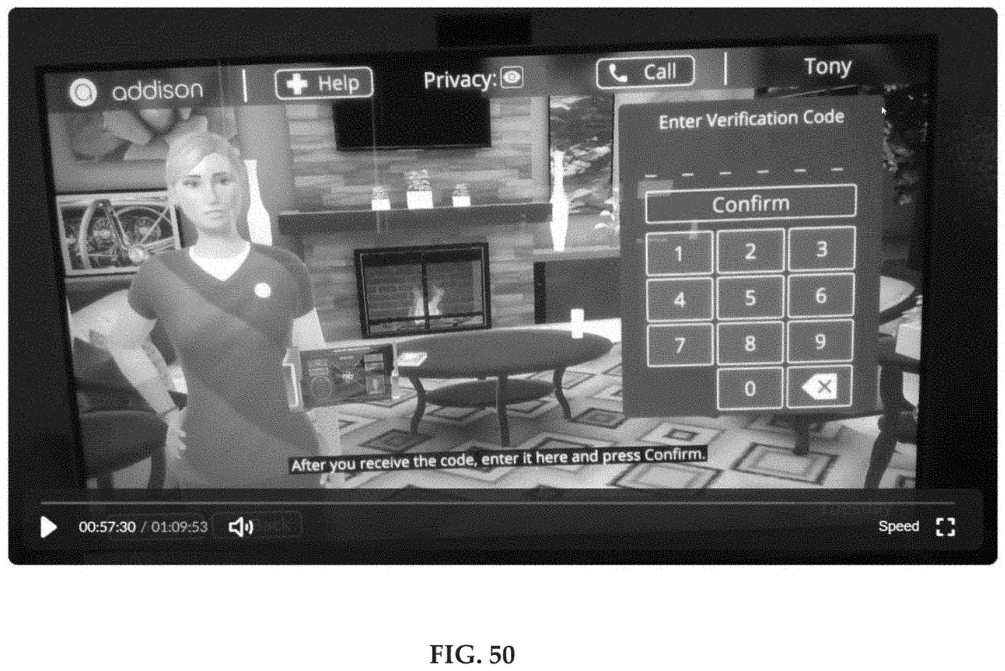

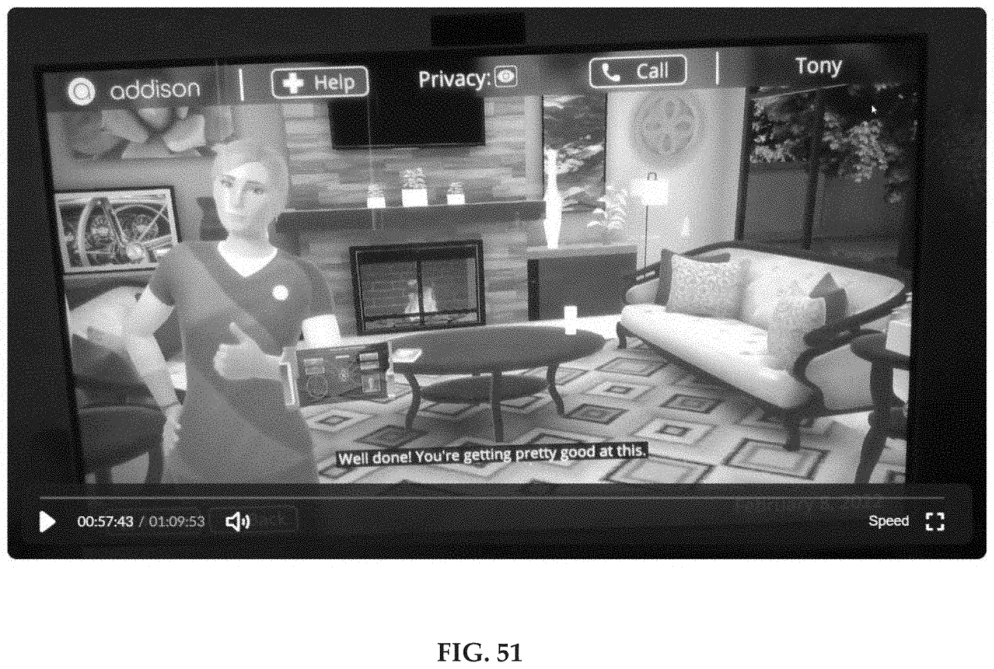

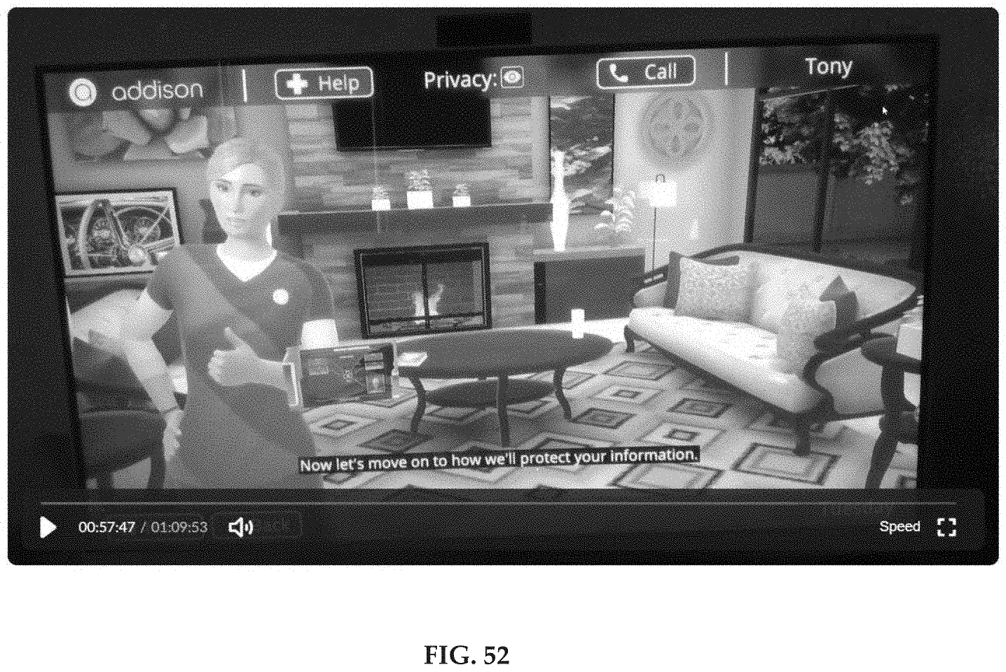

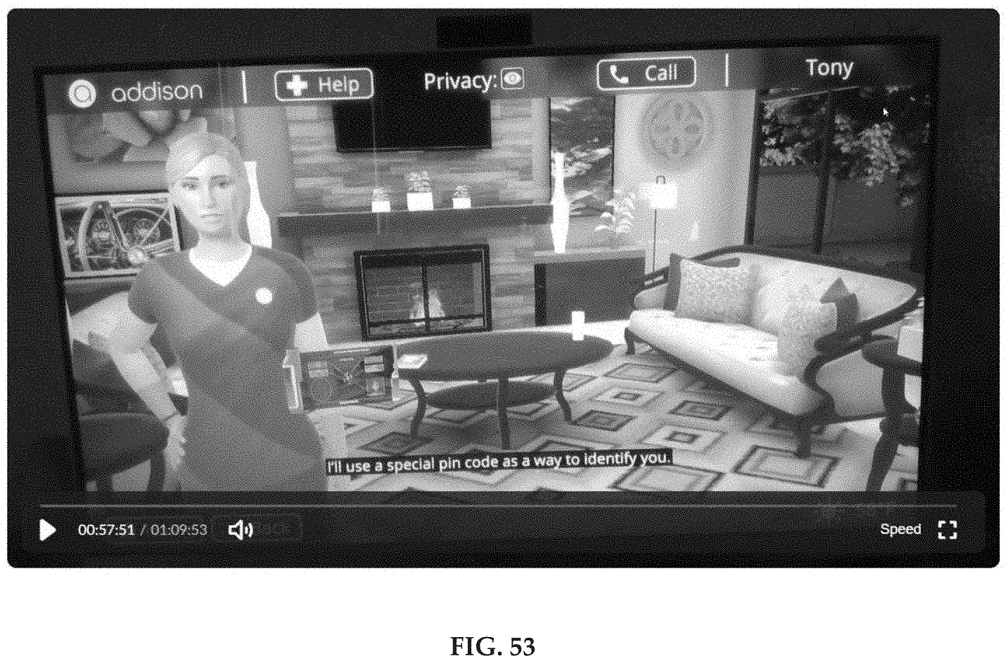

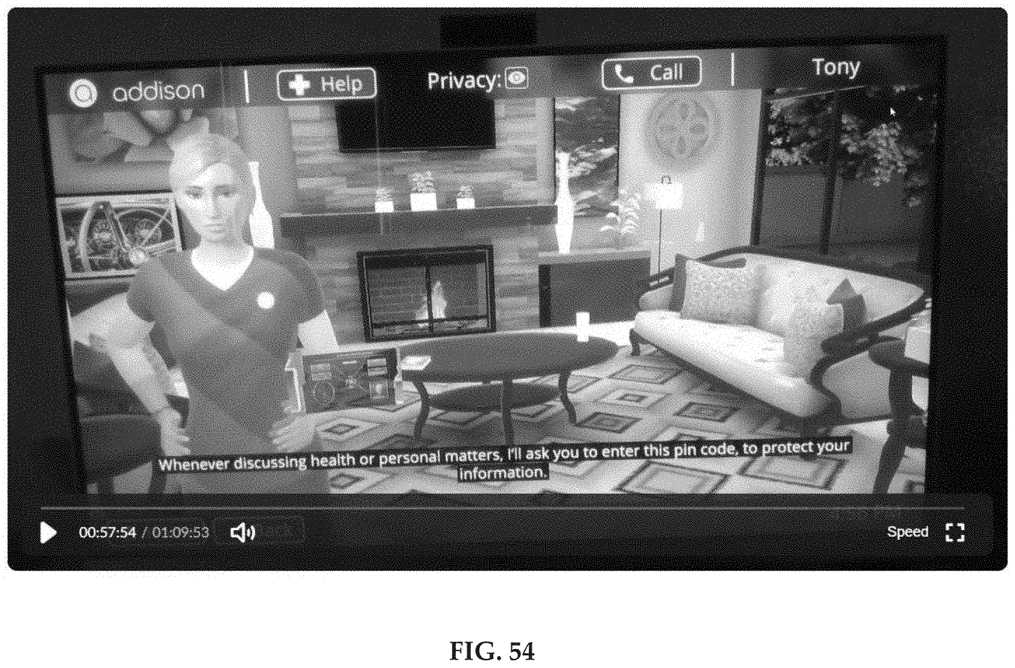

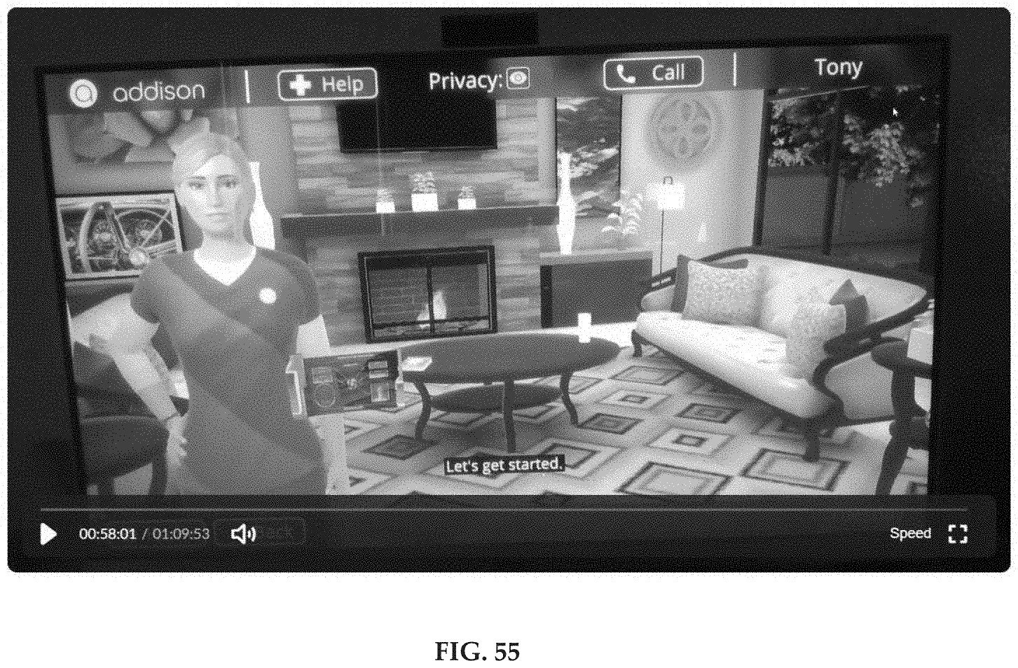

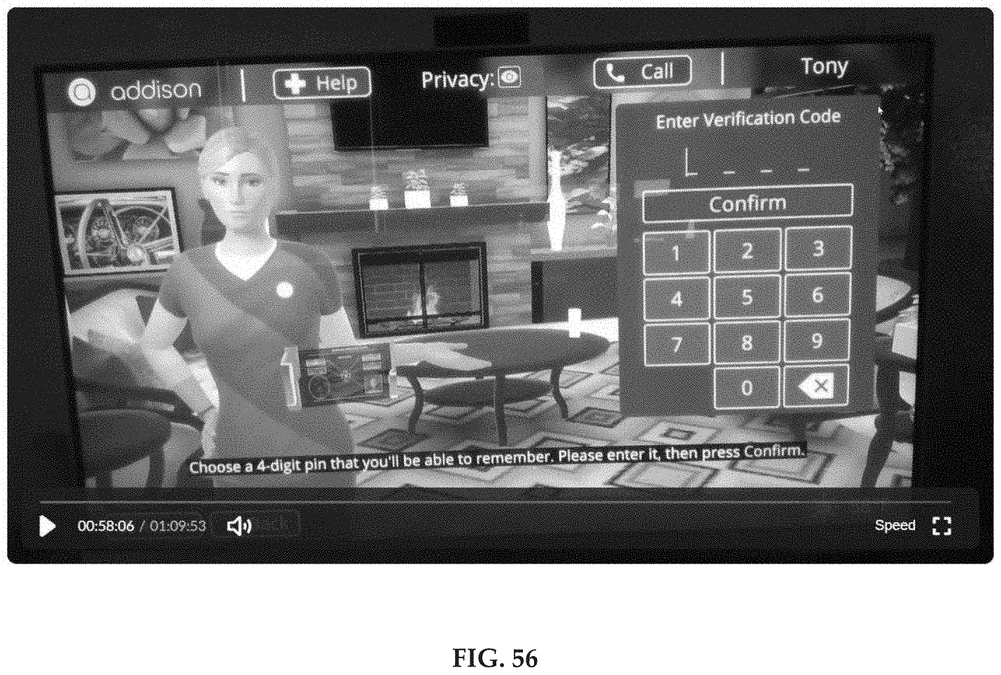

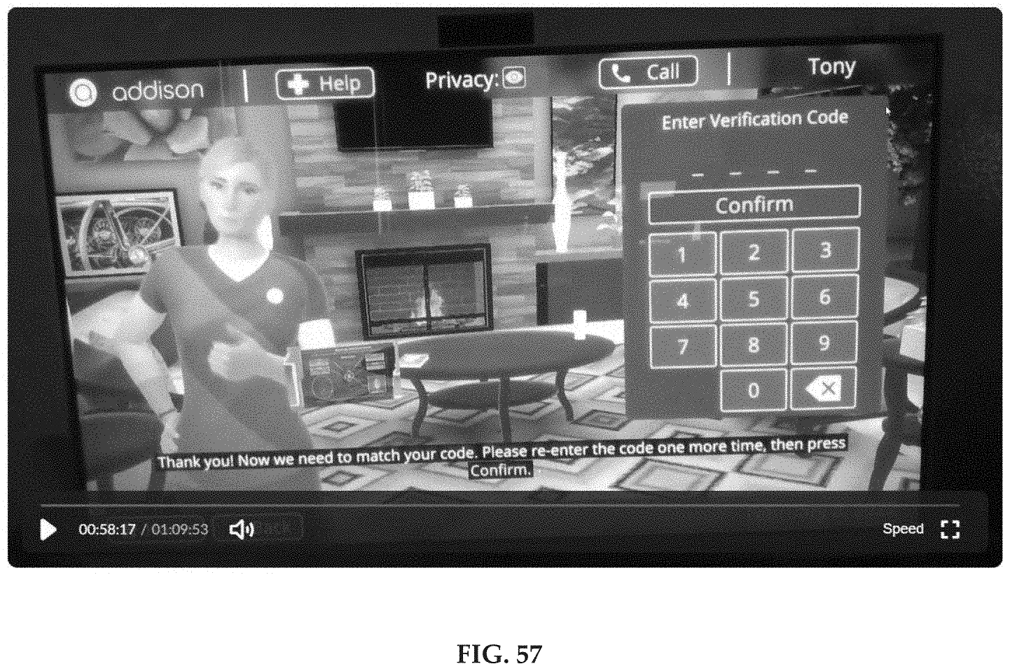

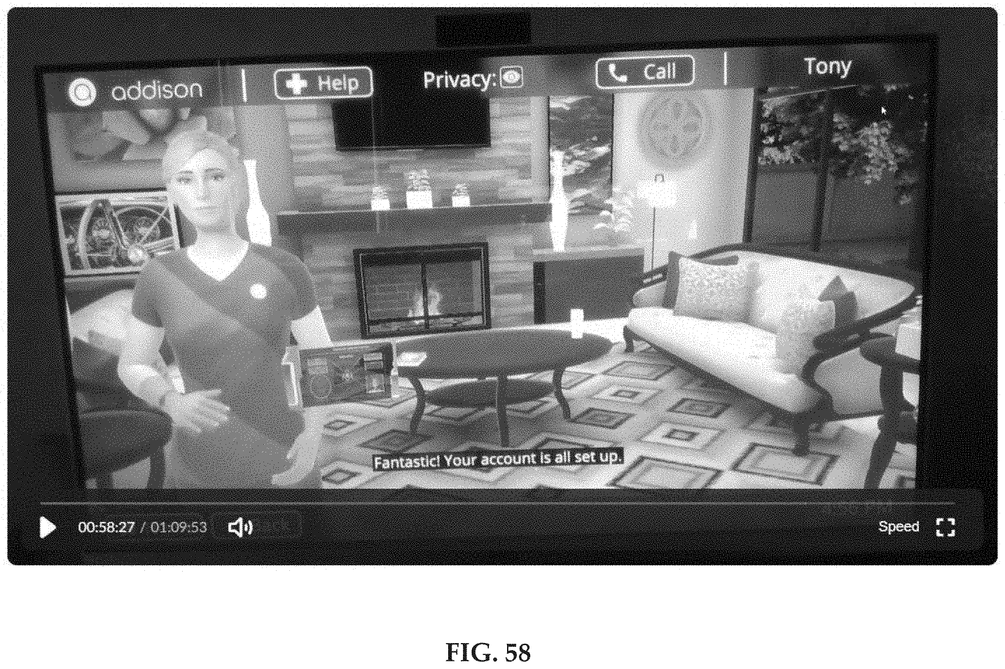

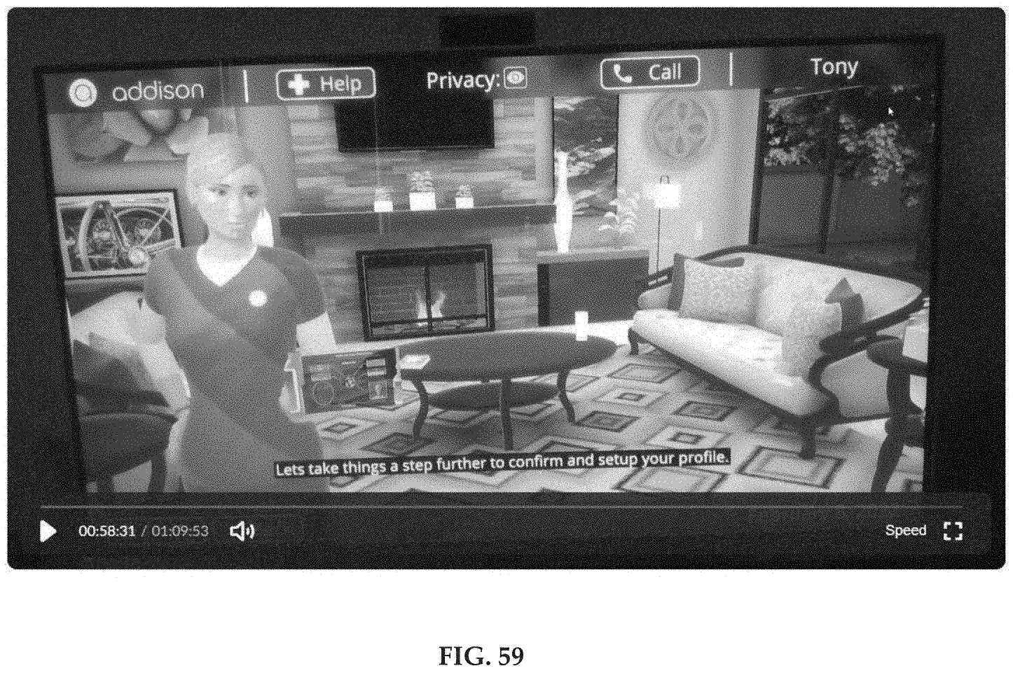

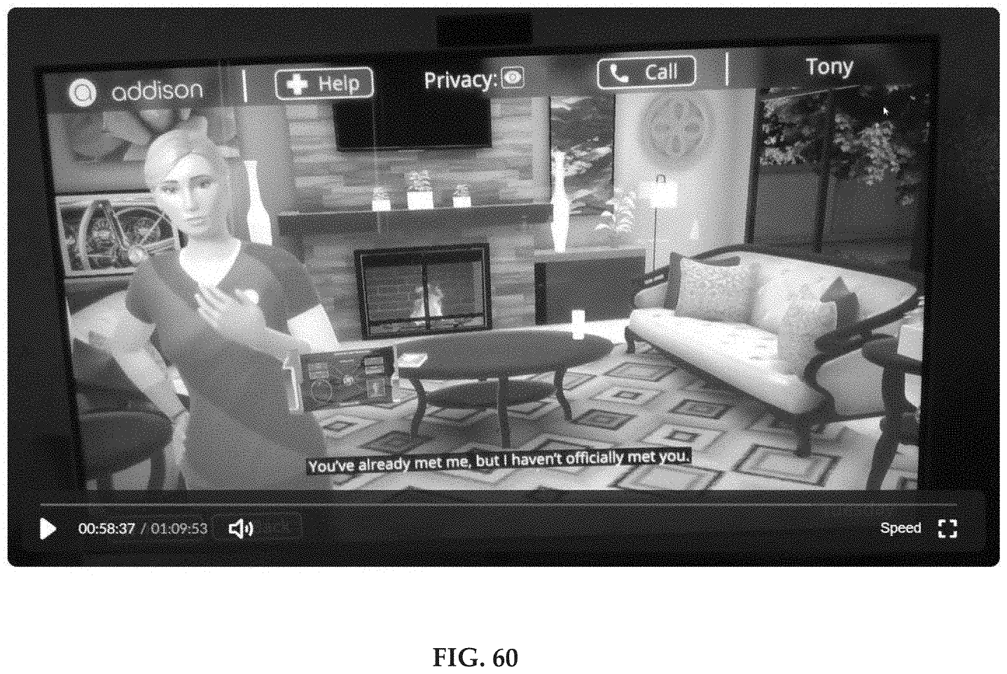

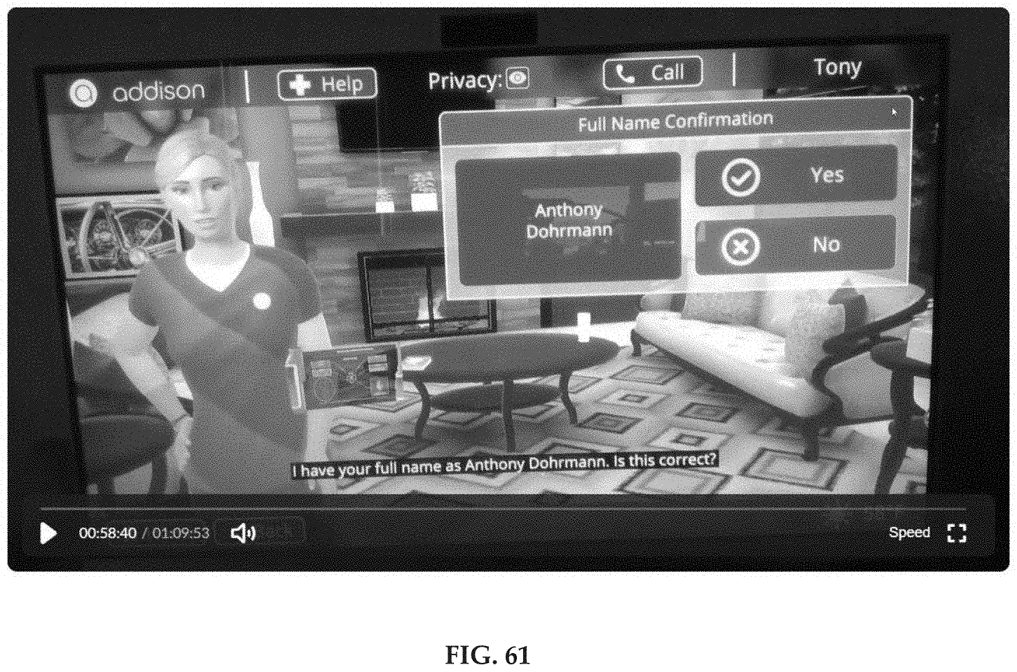

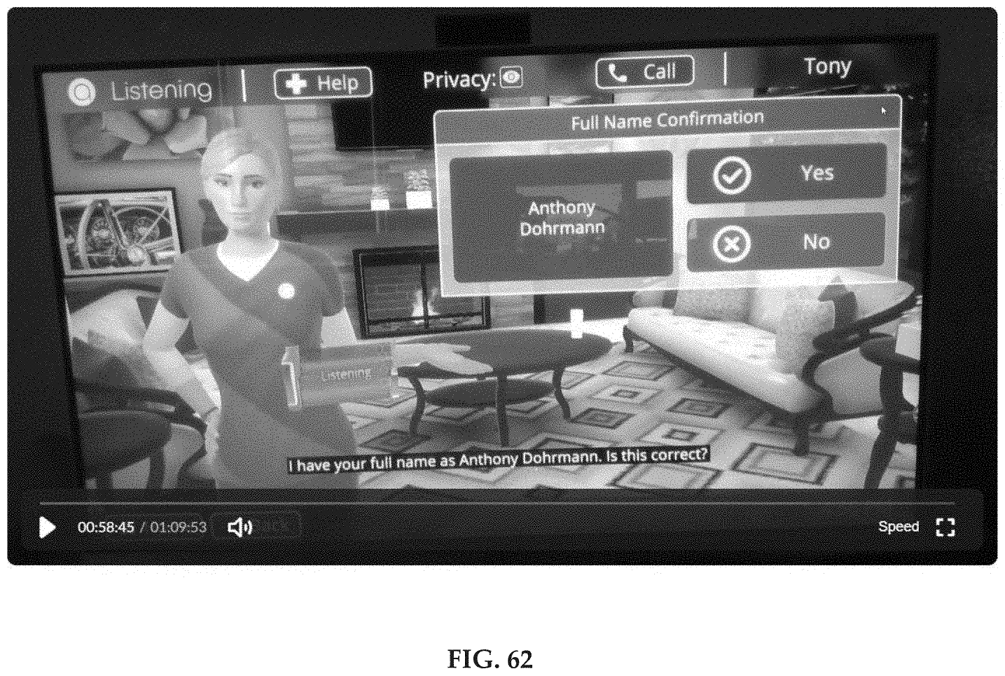

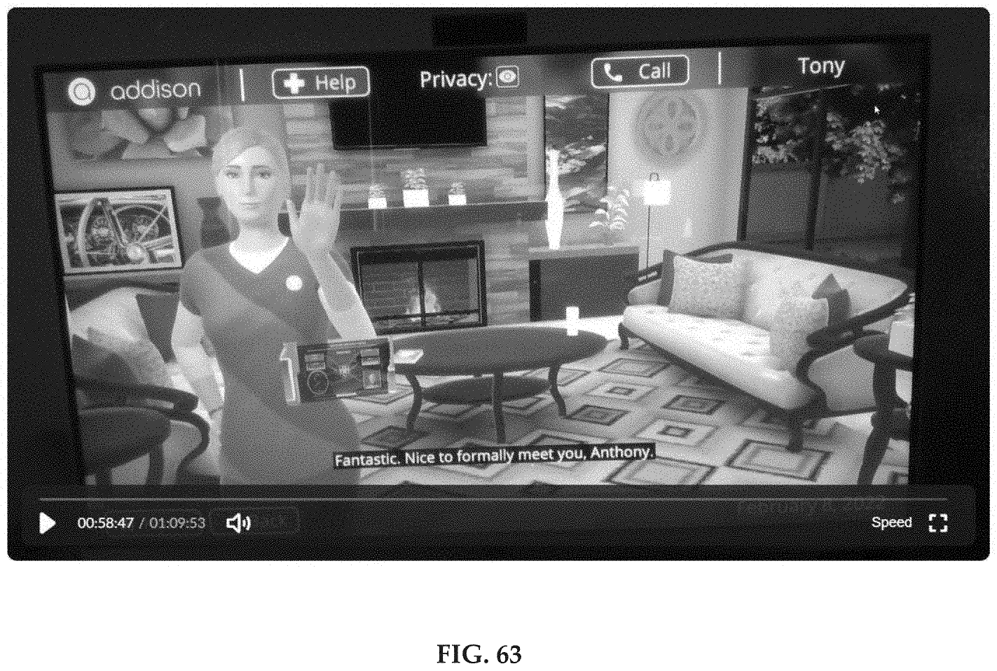

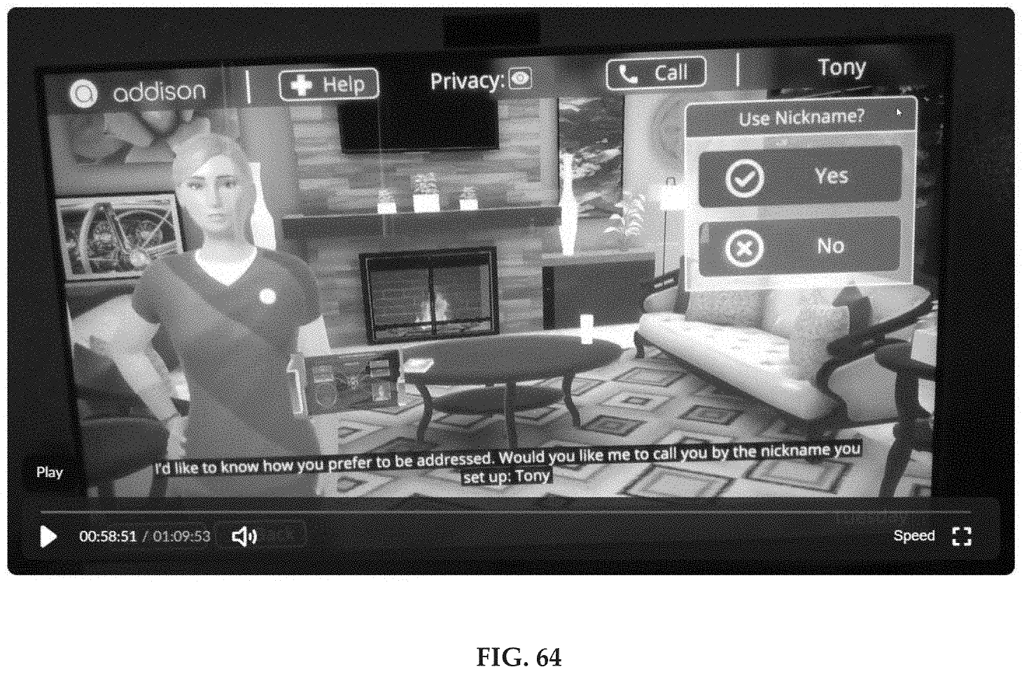

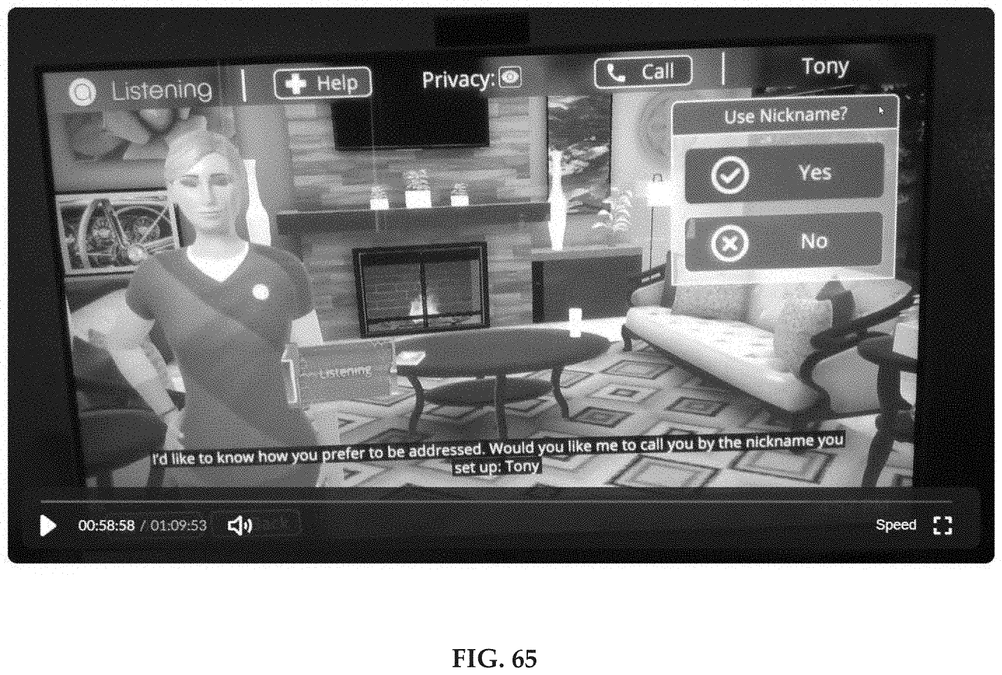

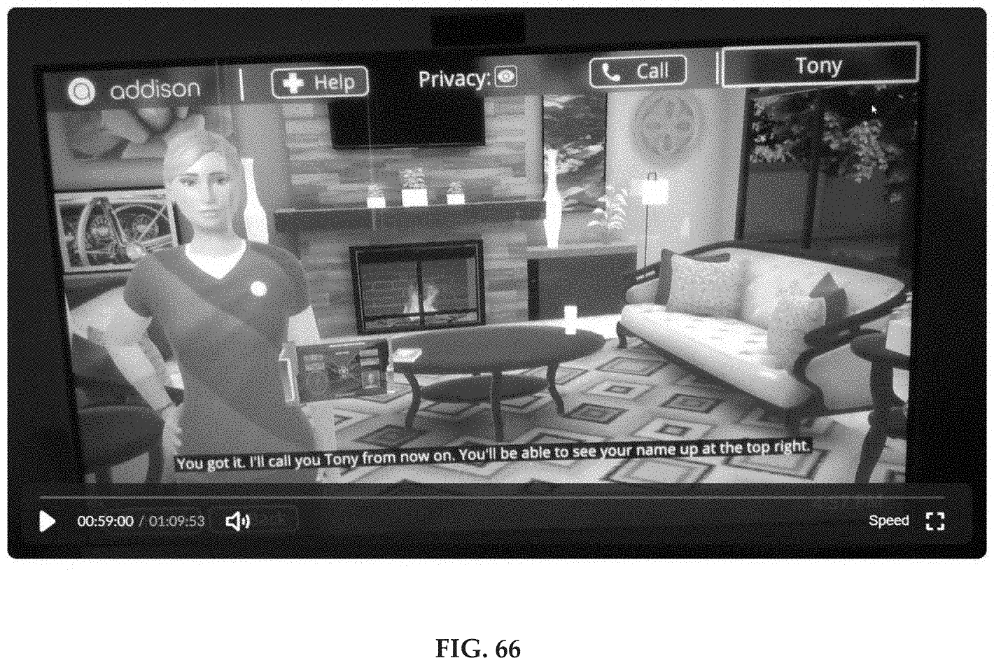

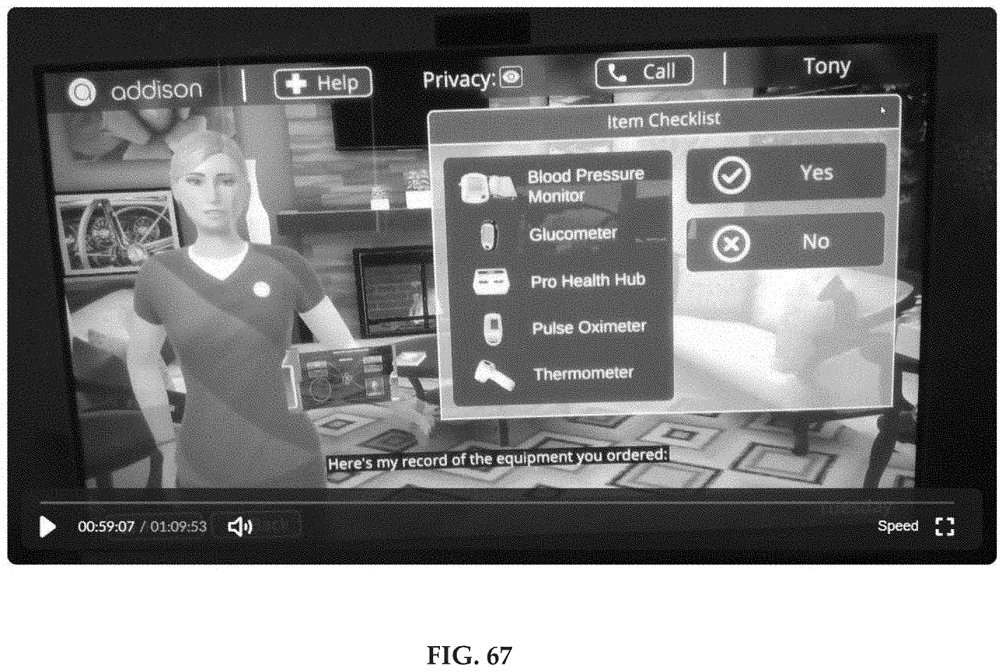

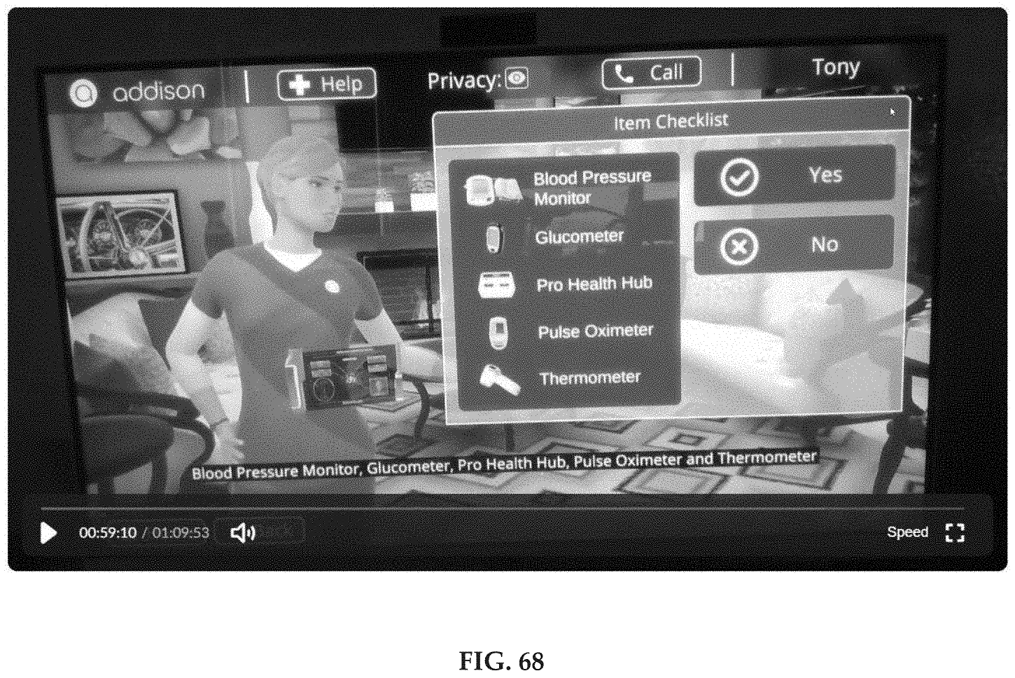

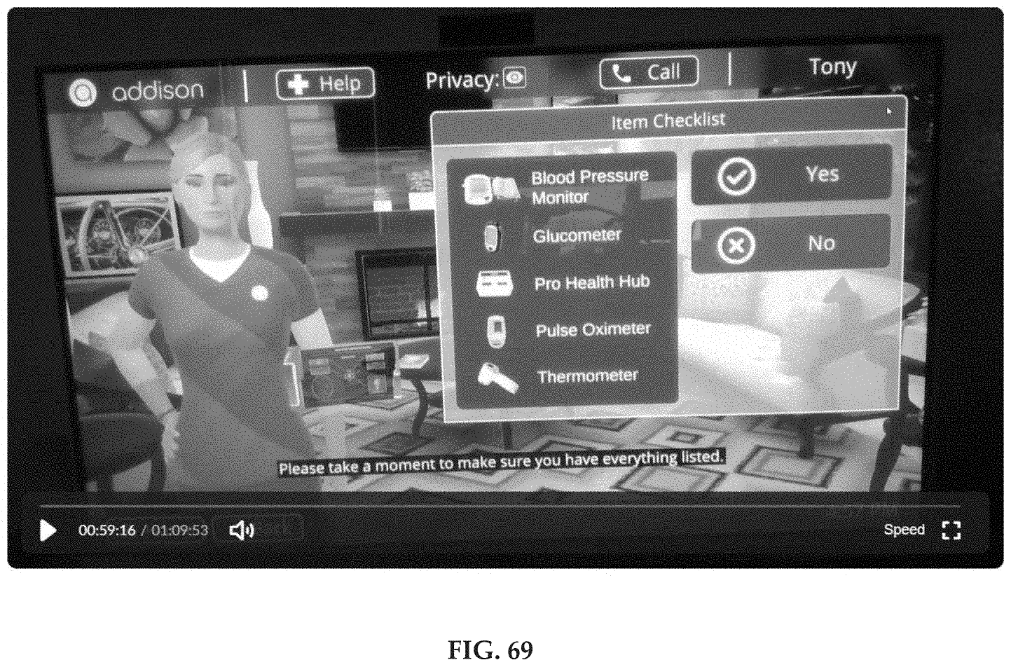

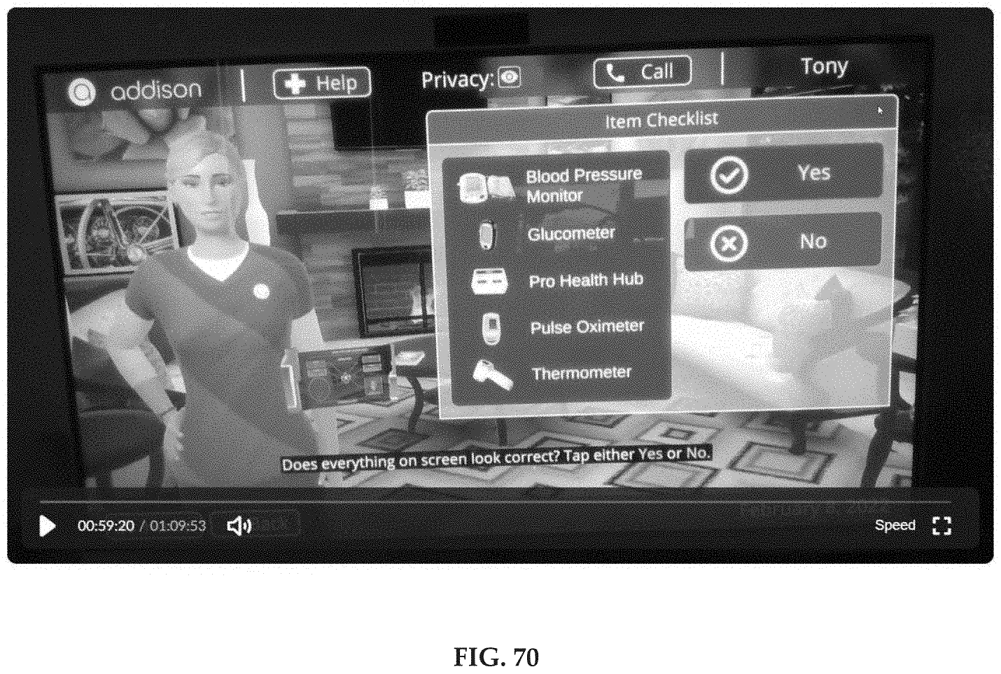

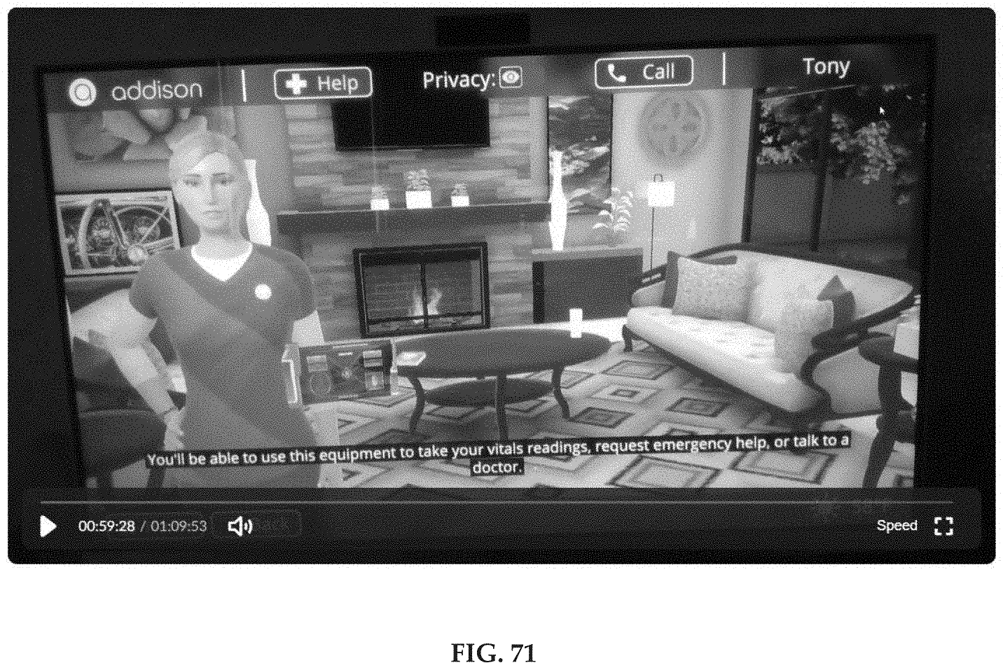

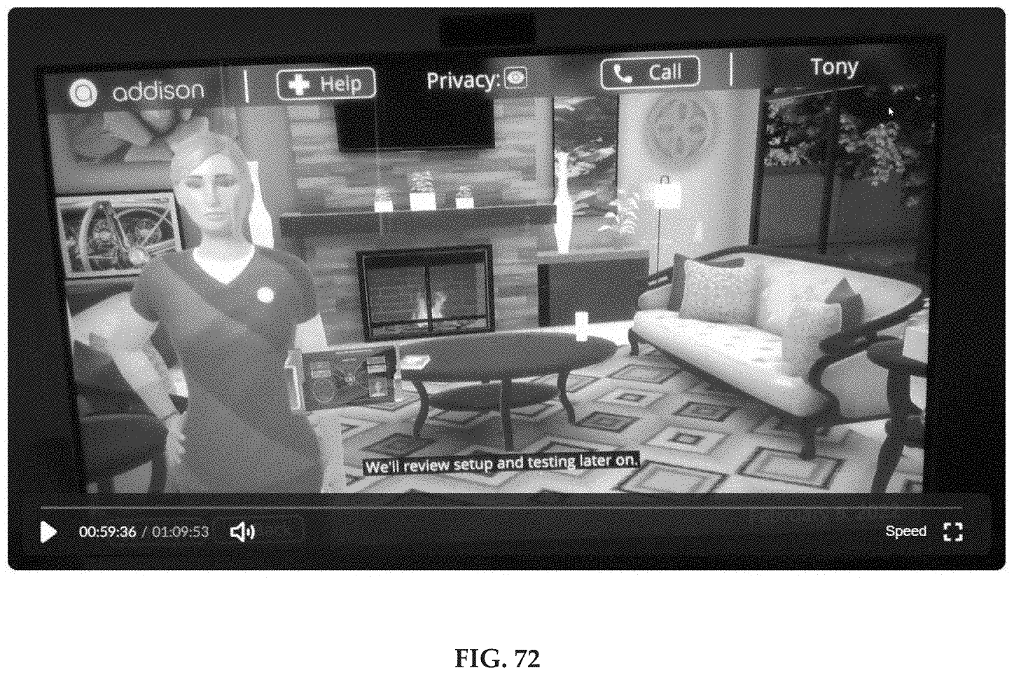

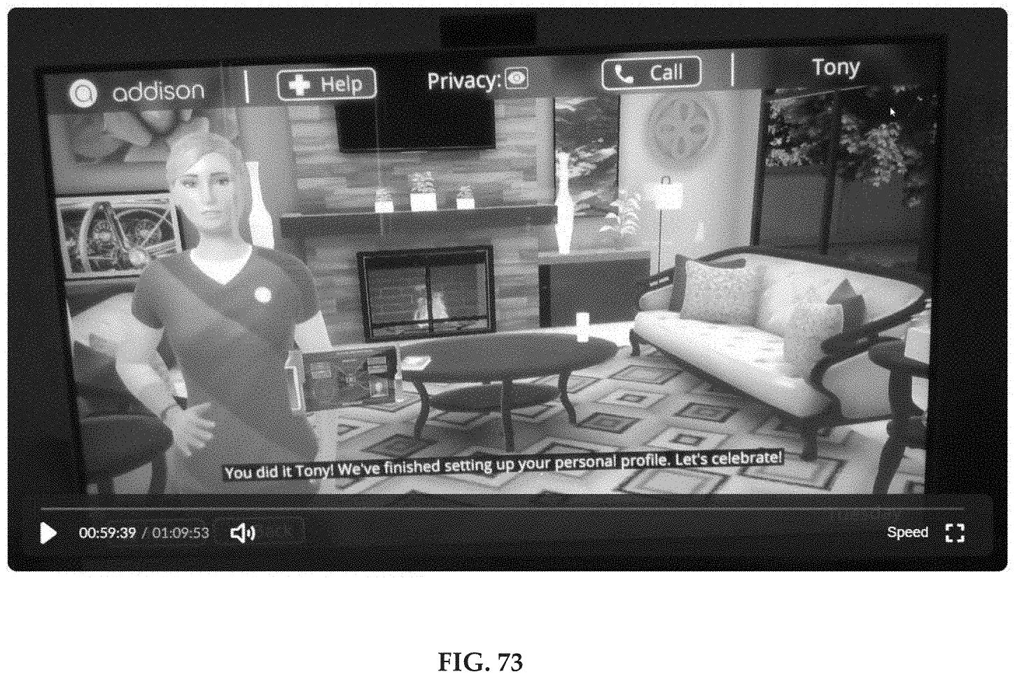

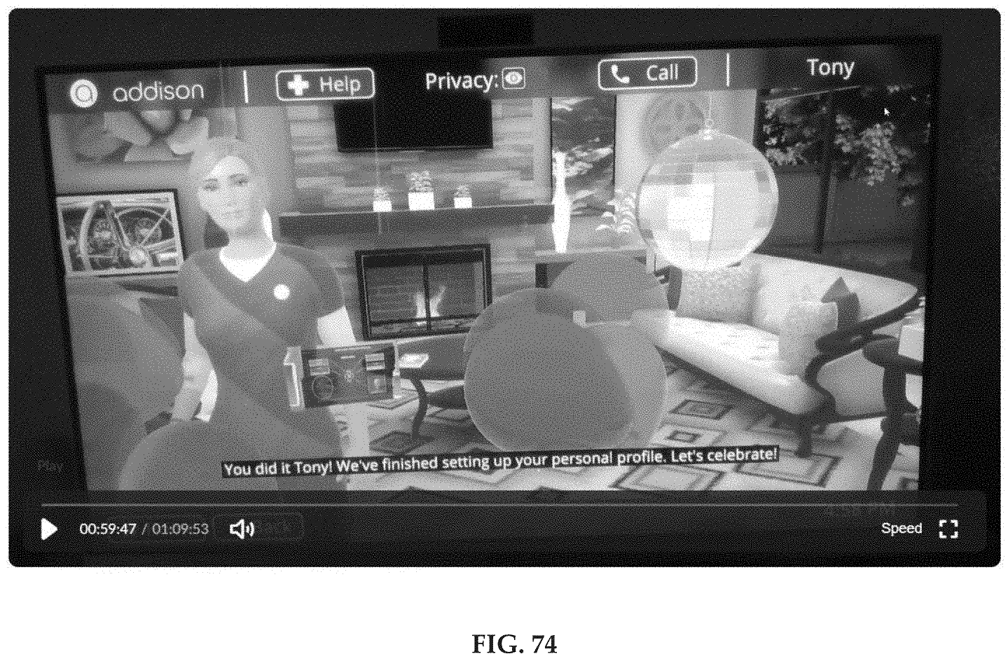

FIGS. 2-74 show exemplary interactive conversational user interfaces.

What follows is an exemplary conversation over the interactive conversational user interface with Addison.

FIG. 2, Addison Tutorial is activated.

FIG. 3, “Hi, I'm Addison. Welcome to Addison care, your personalized health and wellness experience. I'm so excited to get started.”

FIG. 4, “You're taking the first steps towards a healthier life. I can help you with your medication schedule, checking vitals, emergency response and so much more.”

FIG. 5, “Since you're welcoming me into your home, I'll also welcome you into mine.”

FIG. 6, “This is my living room. I'll change locations periodically. I might be in my kitchen, bedroom, or even my backyard!”

FIG. 7, “Let's go over where everything is and how it all works.”

FIG. 8, “I'll always be here on the left side of the screen.”

FIG. 9, “My captions are located at the bottom of the screen. Everything I say will be shown down here. Check it out.”

FIG. 10, “Bamboozle. Cattywampus. Tarnation. Okay, enough fun!”

FIG. 11, “At the bottom right you'll see the time, day, date, and weather.”

FIG. 12, “Now let me show you how to interact with me.”

FIG. 13, “Buttons will appear on the right hand side of the screen.”

FIG. 14, “Like this! The title above these buttons will tell you which screen or menu you're looking at.”

FIG. 15, “Let's try out a button press. Go ahead and tap button.”

FIG. 16, “Perfect. You can also give me voice commands. To do that. Just say Addison and look for my tablet and logo to change color and chime.”

FIG. 17, “Then you can give me a command. You can give me any command that's written out on a button. Let's try it out. Say Addison then say favorite color.”

FIG. 18, unknown speaker, “Addison, favorite color?”

FIG. 19, “Great job! Sometimes I'll ask you a follow up question. And you just have to respond with either your voice or by pressing the button. Let's try it out.”

FIG. 20, “What's your favorite color?”

FIG. 21, unknown speaker, “Purple.”

FIG. 22, “Purple. I like purple too. Great choice. Your natural at this.”

FIG. 23, “Moving on. Let's talk about privacy mode.”

FIG. 24, If you want some privacy, you can touch the privacy switch or say Addison privacy on when it's on, I won't be able to hear you and my visual sensor will be turned off.”

FIG. 25, “Try it out. Tap the privacy switch. Or say privacy on.”

FIG. 26, unknown speaker, “Privacy on.”

FIG. 27, “You'll notice that the privacy button is switched and my tablet is red too. I'll keep myself busy when privacy is on, like listening to my music!”

FIG. 28, “While in privacy mode, I won't be able to hear or see anything. My buttons still work, but I won't be able to hear your voice.”

FIG. 29, “There's one exception, though. for emergency purposes, the only phrase I can always hear is Addison help.”

FIG. 30, “Whether or not I'm in privacy mode, you can say Addison help, or press the Help button, and I'll contact emergency services.”

FIG. 31, “We'll test that an emergency call later, but for now, please press the privacy switch, so we can continue.”

FIG. 32, “Next, let's learn how to contact customer support.”

FIG. 33, “At the top, you'll see a call button. Go ahead and tap or say Addison call to open up the call menu.”

FIG. 34, “Here you'll find several call options. You can press customer support to call directly through the console, or use your phone to dial the number below.”

FIG. 35, “We'll test that a call at a later time, please close the call menu by selecting Close.”

“FIG. 36, “Now, we can review other features in your system. Let's talk about when I'm in idle mode.”

FIG. 37, “When we're not interacting, I'll keep myself busy with random things. I might enjoy TV, a book, or watering my plants.”

FIG. 38, “I pick up new habits all the time. Keep an eye out for all the different things I can do.”

FIG. 39, “We have one additional feature to discuss navigation buttons.”

FIG. 40, “The back button is located down at the bottom left of the screen, and will always take you one step backwards.”

FIG. 41, “To the left of that we have the home button.”

FIG. 42, “This button is an easy way to return to my home screen.”

FIG. 43, “Test it out by tapping or saying home.”

FIG. 44, “There's no place like home. Thank you so much for following along.”

FIG. 45, “Now that we've learned the basics, let's set up your account.”

FIG. 46, “We're going to link your account to this device. So make sure you have access to your email or your phone.”

FIG. 47, “Press the text message or email button. And I'll send you a one time verification code.”

FIG. 48, unknown speaker, “Email.”

FIG. 49, “I'm sending it now. It could take a few moments to receive this code.”

FIG. 50, “After you receive the code, enter it here and press confirm.”

FIG. 51, Well done. You're getting pretty good at this.”

FIG. 52, “Now let's move on to how we'll protect your information.”

FIG. 53, “I'll use a special pin code as a way to identify you.”

FIG. 54, “Whenever discussing health or personal matters. I'll ask you to enter this pin code to protect your information.”

FIG. 55, “Let's get started.”

FIG. 56, “Choose a four-digit PIN that you'll be able to remember. Please enter it, then press confirm.”

FIG. 57, “Thank you! Now we need to match your code. Please re-enter the code one more time. Then press confirm.”

FIG. 58, “Fantastic. Your account is all set up.”

FIG. 59, “Let's take things a step further to confirm and set up your profile.”

FIG. 60, “You've already met me, but I haven't officially met you.”

FIG. 61, “I have your full name as Anthony Dohrmann. Is this correct?”

FIG. 62, unknown speaker, “Yes.”

FIG. 63, “Fantastic. Nice to formally meet you, Anthony.”

FIG. 64, “I'd like to know how you prefer to be addressed. Would you like me to call you by the nickname you set up, Tony?”

FIG. 65, unknown speaker, “Yes.”

FIG. 66, “You got it. I'll call you Tony. From now on. You'll be able to see your name up at the top right.”

FIG. 67, “Here's my record of the equipment you ordered.”

FIG. 68, “Blood Pressure Monitor, Glucometer, Pro Health Hub, Pulse Oximeter and Thermometer.”

FIG. 69, “Please take a moment to make sure you have everything listed.”

FIG. 70, “Does everything on screen look correct? Tap either yes or no.”

FIG. 71, “You'll be able to use this equipment to take your vitals readings, request emergency help, or talk to a doctor.”

FIG. 72, “We'll review setup and testing later on.”

FIG. 73, You did it Tony. We finished setting up your personal profile. Let's celebrate!”

FIG. 74, shows balloons falling.

As illustrated and described herein, exemplary embodiments include Addison having a tablet in hand. It's a point of interaction for her. It lights up when she is listening. She also interacts with the tablet when she is collecting information, such as vital signs. Additionally, other avatars of other ethnicities and/or genders may be utilized.

The system 100 is also configured for two and/or multiple way calling between such parties as health service providers, Addison, family contacts and/or the patient. In many exemplary embodiments, Voice over Internet Protocol (VoIP) is used.

With respect to the networkable health care devices 112, collected data is saved in the system 100 and compared against various metrics and/or thresholds in case health service providers, the patient, and/or family members, etc. need to be notified. In many exemplary embodiments, health service providers may predefine various metrics and/or thresholds in the system 100.

According to various exemplary embodiments, the displayed environment may be automatically and/or manually configured to reflect current weather conditions or typical home configurations for homes within a patient's geographic vicinity. It is designed to be a fully representative experience.

Notably, most exemplary embodiments utilize Unity's eponymous platform. It is used to create two-dimensional, three-dimensional, virtual reality, and augmented reality video games and other simulations. It offers much more flexibility than a browser-based system.

In some exemplary embodiments, Unity can be used to make “digital twins”—virtual copies of real-life objects, environments, and people. Unity can be used to create a non-player character or “NPC.” A NPC is any character (such as in a game) that is not controlled by a player. For example, it applies to characters controlled by a gamemaster or referee rather than by another player. Unity also utilizes artificial intelligence tools in its virtual environments. With a digital twin, Unity can collect synthetic data off the simulation to advance its in real life (“IRL”) twin. Additionally, in various exemplary embodiments, systems may be plugged into Unity, including facial recognition data, location data, CAD data, computer vision data, natural language processing data, blood pressure monitor data, glucometer data, pro health hub data, pulse oximeter data and/or thermometer data. As described and illustrated herein, in many exemplary embodiments, Addison may represent a NPC of an IRL caregiver (or visa versa). Further, Addison may be controlled in part by a patient or other human.

In some exemplary embodiments, the system 100 includes an Emergency Medical System and/or an Emergency Medical Technician module so emergency personnel can immediately access health care information either on site and/or over a network. The system 100 may also be configured to receive and store a Do Not Resuscitate (“DNR”) order and/or a Last Will and Testament, etc.

The system 100 may also be configured with Addison having the ability to track inventories, such as groceries or medicines, and place automatic reorders. The system 100 may be configured with Addison having the ability to order food through various applications or goods or services through vendors such as Amazon®.

In various exemplary embodiments, the system 100 may be configured with facial recognition capabilities for Addison to determine and interpret a patient's face and changes, including mood and/or possible signs of a stroke or cardiovascular event.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. An intelligent secure networked health messaging system configured by at least one processor to execute instructions stored in memory, the system comprising:
a data retention system and a health analytics system, the health analytics system performing asynchronous processing with a user's computing device;
a web services layer providing access to the data retention and the health analytics system;
a batching service, wherein an application server layer transmits a request to the web services layer for data, the request processed by the batching service transparently to the user;
the request processed by the batching service transparently to the user such that the user can continue to use a user-facing unity application without disruption;
the application server layer including a high speed data corridor established between the application server layer and the user's computing device that:
provides the user-facing unity application that accesses the data retention and the health analytics system through the web services layer; and
performs processing based on user interaction with the user-facing unity application, the user-facing unity application configured to execute instructions including:
transmitting an interactive conversational user interface to the user's computing device.

2. The intelligent secure networked health messaging system of claim 1, further comprising the interactive conversational user interface configured to activate a patient tutorial.

3. The intelligent secure networked health messaging system of claim 1, further comprising the interactive conversational user interface configured to display a medication schedule.

4. The intelligent secure networked health messaging system of claim 1, further comprising the interactive conversational user interface configured to display a vital sign.

5. The intelligent secure networked health messaging system of claim 1, further comprising the interactive conversational user interface configured to display an external weather environment similar to a patient's actual external weather environment.

6. The intelligent secure networked health messaging system of claim 1, further comprising the interactive conversational user interface configured with a visual sensor.

7. The intelligent secure networked health messaging system of claim 1, further comprising the interactive conversational user interface configured with a visual sensor for facial recognition of a patient.

8. The intelligent secure networked health messaging system of claim 1, further comprising the interactive conversational user interface configured with a visual sensor for facial recognition of a patient's facial changes.

9. The intelligent secure networked health messaging system of claim 1, further comprising the interactive conversational user interface configured not to receive verbal input.

10. The intelligent secure networked health messaging system of claim 1, further comprising the interactive conversational user interface configured not to receive verbal input unless a patient requests help.

11. The intelligent secure networked health messaging system of claim 1, further comprising the interactive conversational user interface configured to contact an emergency service.

12. The intelligent secure networked health messaging system of claim 1, further comprising the interactive conversational user interface configured to link an account to the system.

13. The intelligent secure networked health messaging system of claim 1, further comprising the interactive conversational user interface configured to display a special pin code.

14. The intelligent secure networked health messaging system of claim 1, further comprising the interactive conversational user interface configured to display a special pin code for entry of information regarding a health matter.

15. The intelligent secure networked health messaging system of claim 1, further comprising the interactive conversational user interface configured to display a special pin code for entry of information regarding a personal matter.

16. The intelligent secure networked health messaging system of claim 1, further comprising the interactive conversational user interface configured to receive information from a blood pressure monitor over a network.

17. The intelligent secure networked health messaging system of claim 1, further comprising the interactive conversational user interface configured to receive information from a glucometer over a network.

18. The intelligent secure networked health messaging system of claim 1, further comprising the interactive conversational user interface configured to receive information from a pulse oximeter over a network.

19. The intelligent secure networked health messaging system of claim 1, further comprising the interactive conversational user interface configured to receive information from a pro health hub over a network.

20. The intelligent secure networked health messaging system of claim 1, further comprising the interactive conversational user interface configured to receive information from a thermometer over a network.
